(19) United States
(12) Patent Application Publication
(10) Pub. No.: US 2025/0000430 A1
(43) Pub. Date: Jan. 2, 2025

(54) DEPRESSION TREATMENT

(71) Applicant: GrayMatters Health Ltd., Haifa (IL)

(72) Inventors: Shai ATTIA, Shimshit (IL); Ran COHEN, Tel-Aviv (IL); Yaakov STERN, Sde Yaakov (IL); Dmitri LAVRO, Kfar Vradim (IL); Oded Shlomo KRAFT-OZ, Haifa (IL)

(73) Assignee: GrayMatters Health Ltd., Haifa (IL)

(21) Appl. No.: 18/882,892

(22) Filed: Sep. 12, 2024

Related U.S. Application Data
(63) Continuation of application No. PCT/IL2023/050261, filed on Mar. 13, 2023.
(60) Provisional application No. 63/319,349, filed on Mar. 13, 2022.

(51) Int. Cl. A61B 5/377; A61B 5/16; A61B 5/375

(52) U.S. Cl. CPC A61B 5/377; A61B 5/165; A61B 5/375

(57) ABSTRACT

A method for training a predetermined subject population expected to experience or is experiencing at least one symptom of depression, including: selecting a population of subjects expected to experience in the future or is experiencing at least one symptom of depression; training the selected population of subjects using a neurofeedback (NF) training protocol to increase an activity of the brain positive valence system, the training comprises, presenting to each subject of the selected population of subjects a sensory indication, instructing the subject to modify the presented sensory indication by performing at least one specific activity, detecting an increase in a brain pleasure response in the subject, and providing to the subject a feedback signal indicating the increase in the brain pleasure response during the presenting.

### RELATED APPLICATIONS

This application is a Continuation of PCT Patent Application No. PCT/IL2023/050261 having International filing date of Mar. 13, 2023, which claims the benefit of priority under 35 USC § 119 (e) of U.S. Provisional Patent Application No. 63/319,349, filed on Mar. 13, 2022. The contents of the above applications are all incorporated by reference as if fully set forth herein in their entirety.

### FIELD AND BACKGROUND OF THE INVENTION

The present invention, in some embodiments thereof, relates to modulating an activity of a mesolimbic brain region and, more particularly, but not exclusively, to modulating an activity of a mesolimbic brain region related to a reward system in a brain.

Depression is one of the most prevalent mental health problems worldwide (Kessler et al., 2009), and result in significant functional impairment and incur tremendous economic costs (Baxter, Vos, Scott, Ferrari, & Whiteford, 2014; Ferrari et al., 2013). Moreover, depression is the second-leading cause of disability, accounting for approximately 8 percent of the global burden of disease (Ferrari et al., 2013). Therefore, development of more efficient treatments, to reduce both prevalence and cost is needed.

### SUMMARY OF THE INVENTION

Some examples of embodiments of the invention are described below (some example of the invention are described herein and an embodiment may include features from more than one example and/or fewer than all features of an example):

Example 1. A neurofeedback method, comprising:
delivering a stimulus to said subject, wherein said stimulus is selected to increase a pleasure response in said subject;
detecting an increase in said pleasure response in said subject;
modifying said stimulus in a way selected to increase said pleasure response in said subject.

Example 2. A method according to example 1, comprising:
repeating said delivering with said modified stimulus.

Example 3. A method according to any one of the previous examples, wherein said detecting comprises detecting an increase in activity of at least one mesolimbic brain region.

Example 4. A method according to example 3, wherein said at least one mesolimbic brain region comprises a brain region related to a reward response in said subject.

Example 5. A method according to any one of examples 3 or 4, wherein said at least one mesolimbic brain region comprises at least one of, the ventral striatum (VS), the Nucleus Accumbens, Ventral tegmental area, OrbitoFrontal cortex, and the Insula.

Example 6. A method according to any one of the previous examples, comprising:
recording during said delivering said detecting and said modifying said stimulus, electrical signals using at least one electrode positioned on a head of a subject.

Example 7. A method according to example 6, wherein said at least one electrode is positioned in at least one location of C3, C4, Cz, FCZ, P3, Pz and P4 locations of an extended 10-20 electroencephalography (EEG) coordinate system.

Example 8. A neurofeedback method, comprising:
delivering a stimulus to said subject, wherein said stimulus is selected to increase a pleasure response in said subject;
detecting a reduction in pleasure response or lack of pleasure response in said subject;
modifying said delivering according to said detecting.

Example 9. A method according to example 8, wherein said modifying said delivering comprises replacing said stimulus with a different stimulus.

Example 10. A method according to example 8, wherein said modifying said delivering comprises modifying at least one parameter of said stimulus.

Example 11. A method according to example 10, wherein said at least one parameter comprises at least one of, intensity of said stimulus, duration of said stimulus, type of said stimulus.

Example 12. A method according to any one of examples 8 to 11, comprising, delivering an indication related to capacity of said subject to increase said pleasure response in response to said delivered stimulation based on said detecting.

Example 13. A method according to any one of examples 8 to 12, wherein said subject is diagnosed with Anhedonia.

Example 14. A neurofeedback method, comprising:
determining an activity level of at least one brain region;
delivering a feedback signal to a subject indicating that said determined activity level has reached a target activation level;
providing an indication to said subject indicating that said target activation level of said at least one brain region is maintained for a pre-determined time period.

Example 15. A method according to example 14, comprising:
instructing a subject to reach a target activation level of at least one brain region.

Example 16. A method according to any one of examples 14 or 15, comprising:
instructing said subject to maintain said target activation level of said at least one brain region for a pre-determined time period.

Example 17. A neurofeedback method, comprising:
delivering a stimulus to said subject, wherein said stimulus is selected to increase a pleasure response in said subject;
determining an activity level of at least one brain region during said delivering;
detecting that said subject does not appreciate said increase in said pleasure response;
generating an indication according to said detecting.

Example 18. A neurofeedback method according to example 17, wherein said detecting comprises detecting that said subject does not appreciate said increase in said pleasure response using a p300 algorithm and/or EFP.

Example 19. A system for delivering of a neurofeedback treatment, comprising:
a patient interface configured to deliver at least one human detectable indication to a subject;
a control unit, comprising:
a memory;
an EEG recording unit, configured to receive electrical signals from one or more electrodes positioned on a head of a subject;
a control circuitry, wherein said control circuitry is configured to:
signal said patient interface to deliver a stimulus stored in said memory to a subject, wherein said stimulus is selected to increase a pleasure response in said subject;
measure EEG signals according to the electrical signals received by said EEG recording unit;
detect an increase in activity in at least one mesolimbic brain regions using at least one algorithm and/or an electrical fingerprint (EFP) stored in said memory, wherein said detected increase in activity indicates an increase in said pleasure response in said subject;
modify said stored stimulus in a way selected to increase said pleasure response in said subject; and
signal said patient interface to deliver said modified stimulus to said subject.

Below are some additional examples of embodiments of the invention (some example of the invention are described herein and an embodiment may include features from more than one example and/or fewer than all features of an example):

Example 1. A method for training a predetermined subject population expected to experience or is experiencing at least one symptom of depression, comprising:
selecting a population of subjects expected to experience in the future or is experiencing at least one symptom of depression;
training said selected population of subjects using a neurofeedback (NF) training protocol to increase an activity of the brain positive valence system, said training comprises, presenting to each subject of said selected population of subjects a sensory indication, instructing said subject to modify said presented sensory indication by performing at least one specific activity, detecting an increase in a brain pleasure response in said subject, and providing to said subject a feedback signal indicating said increase in said brain pleasure response during said presenting.

Example 2. A method according to example 1, wherein said at least one symptom of depression comprises depressed mood and/or anhedonia.

Example 3. A method according to any one of examples 1 or 2, wherein said at least one symptom of depression comprises at least one of, weight loss, weight gain, insomnia, hypersomnia, psychomotor agitation, psychomotor retardation, reduction in energy, worthlessness, guilt, concentration, suicidal thoughts and/or suicidal behaviors.

Example 4. A method according to any one of the previous examples, wherein said selecting comprises selecting a population of subjects having a first degree relative previously diagnosed with major depression or selecting a population of subjects which are a first responders.

Example 5. A method according to any one of examples 1 to 3, wherein said selecting comprises selecting a population of subjects diagnosed with at least one of, post-traumatic stress disorder (PTSD), attention deficit hyperactivity disorder (ADHD), substance-induced mood disorder, chronic kidney disease (CKD) and/or Premenstrual dysphoric disorder (PMDD).

Example 6. A method according to any one of examples 1 to 3, wherein said selecting comprises selecting a population of subjects in a risk for developing peripartum depression or already diagnosed with peripartum and/or postpartum depression.

Example 7. A method according to any one of examples 1 to 3, wherein said selecting comprises selecting a population of subjects in a risk for developing seasonal affective disorder (SAD) or that was previously diagnosed with SAD.

Example 8. A method according to any one of examples 1 to 3, wherein said selecting comprises selecting a population of subjects diagnosed with cancer.

Example 9. A method according to example 8, wherein said cancer comprises pancreatic cancer and/or lung cancer.

Example 10. A method according to any one of the previous examples, wherein said sensory indication comprises a visual and/or an audio indication, and wherein said providing comprises providing said feedback signal by modifying said visual and/or audio indication.

Example 11. A method according to example 10, wherein said providing comprises providing said feedback signal by modifying said visual and/or audio indication in a way selected to increase said brain pleasure response in said subject.

Example 12. A method according to example 11, comprising repeating said presenting with said modified visual and/or audio indication.

Example 13. A method according to any one of the previous examples, wherein said presented sensory indication is selected to increase a pleasure response in said subject.

Example 14. A method according to any one of the previous examples, wherein said detecting comprises measuring EEG signals from one or more electrodes positioned on a subject head during said presenting and said detecting, and wherein said detecting comprises detecting said increase in said brain pleasure response based on said measured EEG signals.

Example 15. A method according to any one of the previous examples, wherein said detecting comprises detecting an increase in activity of at least one mesolimbic brain region.

Example 16. A method according to example 15, wherein said at least one mesolimbic brain region comprises a brain region related to a reward response in said subject.

Example 17. A method according to any one of examples 15 or 16, wherein said at least one mesolimbic brain region comprises at least one of, a ventral striatum (VS), a Nucleus Accumbens, a Ventral tegmental area, an Orbitofrontal cortex, and an Insula of the subject.

Example 18. A method according to any one of the previous examples, comprising:
providing instructions to said subject to perform said at least one specific activity when experiencing in the future said at least one depression symptom.

Example 19. A method according to any one of the previous examples, wherein said at least one specific activity comprises a cognitive activity or a mental activity, selected to increase said brain pleasure response in said subject.

Example 20. A method according to any one of the previous examples, wherein said training comprises training said subject using said NF training protocol in two or more training sessions performed in a time difference of at least 12 hours therebetween.

Example 21. A method according to any one of the previous examples, comprising modifying said presented sensory indication or selecting a sensory indication to be presented to said subject, according to said selected subject and prior to said presenting.

Example 22. A neurofeedback method, comprising:
delivering a stimulus to a subject, wherein said stimulus is selected to indicate and/or increase a pleasure response in a brain of said subject;
detecting an increase in said pleasure response in said subject brain;
modifying said stimulus in a way selected to increase said pleasure response in said subject.

Example 23. A method according to example 22, comprising:
repeating said delivering with said modified stimulus.

Example 24. A method according to any one of examples 22 or 23, wherein said detecting comprises detecting an increase in activity of at least one mesolimbic brain region.

Example 25. A method according to example 24, wherein said at least one mesolimbic brain region comprises a brain region related to a reward response in said subject.

Example 26. A method according to any one of examples 24 or 25, wherein said at least one mesolimbic brain region comprises at least one of, the ventral striatum (VS), the Nucleus Accumbens, Ventrall tegmental area, OrbitoFrontal cortex, and the Insula.

Example 27. A method according to any one of examples 22 to 26, comprising:
recording during said delivering, said detecting and said modifying said stimulus, electrical signals using at least one electrode positioned on a head of a subject.

Example 28. A method according to example 27, wherein said at least one electrode is positioned in at least one location of C3, C4, Cz, FCZ, P3, Pz and P4 locations of an extended 10-20 electroencephalography (EEG) coordinate system.

Example 29. A neurofeedback method, comprising:
delivering a stimulus to said subject, wherein said stimulus is selected to indicate and/or increase a pleasure response in said subject;
detecting a reduction in pleasure response or lack of pleasure response in said subject;
modifying said delivering according to said detecting.

Example 30. A method according to example 29, wherein said modifying said delivering comprises replacing said stimulus with a different stimulus.

Example 31. A method according to example 29, wherein said modifying said delivering comprises modifying at least one parameter of said stimulus.

Example 32. A method according to example 31, wherein said at least one parameter comprises at least one of, intensity of said stimulus, duration of said stimulus, type of said stimulus.

Example 33. A method according to any one of examples 29 to 32, comprising, delivering an indication related to capacity of said subject to increase said pleasure response in response to said delivered stimulation based on said detecting.

Example 34. A method according to any one of examples 29 to 33, wherein said subject is diagnosed with Anhedonia.

Example 35. A neurofeedback method, comprising:
determining an activity level of at least one brain region;
delivering a feedback signal to a subject indicating that said determined activity level has reached a target activation level;
providing an indication to said subject indicating that said target activation level of said at least one brain region is maintained for a pre-determined time period.

Example 36. A method according to example 35, comprising:
instructing a subject to reach said target activation level of said at least one brain region.

Example 37. A method according to any one of examples 35 or 36, comprising:
instructing said subject to maintain said target activation level of said at least one brain region for a pre-determined time period.

Example 38. A neurofeedback method, comprising:
delivering a stimulus to said subject, wherein said stimulus is selected to indicate and/or increase a pleasure response in said subject;
determining an activity level of at least one brain region during said delivering;
detecting that said subject does not appreciate said increase in said pleasure response;
generating an indication according to said detecting.

Example 39. A neurofeedback method according to example 38, wherein said detecting comprises detecting that said subject does not appreciate said increase in said pleasure response using a p300 algorithm.

Example 40. A method to treat a predetermined patient population for major depression, the method comprising:
selecting a population of patients diagnosed with major depression;
delivering a NF treatment to said population;
achieving a reduction of at least 3 points in average in a Snaith-Hamilton pleasure scale modified for clinician administration (SHAPS-C) in said selected population, after a time period of at least 5 weeks of said delivering.

Example 41. A method according to example 40, wherein said delivering comprises training said selected population using said NF treatment to increase a pleasure response in the brain and/or to increase activity of a positive valence system of the brain, when exposed to at least one sensory indication.

Example 42. A method according to any one of examples 40 or 41, wherein said delivering comprises training said selected population using said NF treatment to modulate an activity of at least one mesolimbic brain region or at least one biomarker thereof, when exposed to at least one sensory indication.

Example 43. A method according to example 42, wherein said delivering said NF treatment comprises:
recording EEG signals from said selected population during said exposure to said at least one sensory indication, extracting one or more signals which selectively indicate an activity level of said at least one mesolimbic brain region or biomarker thereof, from said recorded EEG signals, and providing feedback to said population regarding an activity of said at least one mesolimbic brain region, or said at least one biomarker thereof, based on the one or more extracted signals.

Example 44. A method according to any one of examples 40 to 42, wherein said delivered NF treatment is a functional magnetic resonance imaging (fMRI) neurofeedback treatment.

Example 45. A method to treat a predetermined patient population for major depression, the method comprising:
selecting a population of patients diagnosed with major depression;
delivering a NF treatment to said population;
achieving a reduction of at least 7 points in average in a Hamilton Depression Rating Scale (HDRS) in said selected population, after a time period of at least 5 weeks of said delivering.

Example 46. A method according to example 45, wherein said delivering comprises training said selected population using said NF treatment to increase a pleasure response in the brain and/or to increase activity of a positive valence system of the brain, when exposed to at least one sensory indication.

Example 47. A method according to any one of examples 45 or 46, wherein said delivering comprises training said selected population using said NF treatment to modulate an activity of at least one mesolimbic brain region or at least one biomarker thereof, when exposed to at least one sensory indication.

Example 48. A method according to example 47, wherein said delivering said NF treatment comprises:
recording EEG signals from said selected population during said exposure to said at least one sensory indication, extracting one or more signals which selectively indicate an activity level of said at least one mesolimbic brain region or biomarker thereof, from said recorded EEG signals, and providing feedback to said population regarding an activity of said at least one mesolimbic brain region, or said at least one biomarker thereof, based on the one or more extracted signals.

Example 49. A method according to any one of examples 45 to 47, wherein said delivered NF treatment is a functional magnetic resonance imaging (fMRI) neurofeedback treatment.

Example 50. A method for neurofeedback assessment, comprising:
selecting a population of subjects currently experiencing or expected to experience in the future, at least one symptom of depression;
presenting using a user interface of a system, to each subject of said selected population of subjects, a sensory indication configured to indicate or induce a pleasure response in the subject brain;
instructing said subject using said user interface to modify said presented sensory indication by performing at least one specific activity;
measuring by a control circuitry electrical signals from said subject brain;
detecting by said control circuitry an increase in said brain pleasure response in said subject based on said measured electrical signals, and providing during said presenting, to said subject by said user interface a feedback signal indicating said increase in said brain pleasure response.

Example 51. A drug for use in the treatment of depression, wherein said drug is administered before and/or after a neurofeedback training.

Example 52. A drug according to example 51, wherein said drug belongs to a family of Selective Serotonin Reuptake Inhibitors (SSRI), or to a family of Serotonin and Norepinephrine Reuptake Inhibitors (SNRI), or to a family of Tricyclic and Tetracyclic Antidepressants, or to a family of Atypical Antidepressants, or to a family of N-methyl D-aspartate (NMDA) Antagonists.

Example 53. A drug according to any one of examples 51 or 52, wherein said drug is administered before and/or after a neurofeedback training that includes:
presenting a sensory indication configured to indicate or induce a pleasure response in a subject brain;
delivering instructions to modify said presented sensory indication by performing at least one specific activity;
measuring electrical signals from a brain;
detecting an increase in a brain pleasure response based on said measured electrical signals, and providing a feedback signal indicating said increase in said brain pleasure response.

Example 54. A system for delivery of a neurofeedback (NF) training, comprising:
a trainee interface configured to deliver at least one human detectable indication to a subject; a control unit, comprising:
a memory;
an EEG recording unit, configured to receive electrical signals from one or more electrodes positioned on a head of a subject;
a control circuitry, wherein said control circuitry is configured to:
signal said trainee interface to deliver a sensory indication stored in said memory to a subject, wherein said sensory indication is selected to increase a pleasure response in said subject brain;
measure EEG signals according to the electrical signals received by said EEG recording unit;
detect an increase in activity of at least one mesolimbic brain region based on said measured EEG signals using at least one algorithm stored in said memory, wherein said detected increase in activity indicates an increase in said pleasure response in said subject;
modify said sensory indication in a way selected to increase said pleasure response in said subject;
and
signal said trainee interface to deliver said modified sensory indication to said subject.

Example 55. A system according to example 54, wherein said control circuitry is configured to deliver a human detectable indication to said subject using said trainee interface with instructions to modify said sensory indication.

Example 56. A system according to any one of examples 54 or 55, wherein said sensory indication comprises a visual and/or audio interface.

Example 57. A system according to any one of examples 54 to 56, wherein said detected increase in activity in said at least one mesolimbic brain region is a relative increase in activity compared to one or more other brain regions.

Example 58. A system according to any one of examples 54 to 57, wherein said at least one mesolimbic brain region comprises at least one of, a ventral striatum (VS), a Nucleus Accumbens, a Ventral tegmental area, an Orbitofrontal cortex, and an Insula of the subject.

Example 59. A system according to any one of examples 54 to 58, wherein said control circuitry is configured to:
determine an activity level of said at least one mesolimbic brain region based on said measured EEG signals;
signal said trainee interface to deliver at least one feedback signal to said subject indicating that said determined activity level has reached a target activation level and that the target activation level is maintained for a pre-determined time period.

Example 60. A system according to example 59, wherein said control circuitry is configured to deliver a human detectable indication to said subject using said trainee interface with instructions to reach a target modification of said sensory indication or to reach said target activation level of said at least one mesolimbic brain region.

Example 61. A system according to any one of examples 59 or 60, wherein said control circuitry is configured to deliver a human detectable indication to said subject using said trainee interface with instructions to reach a target modification of said sensory indication or to reach said target activation level of said at least one mesolimbic brain region.

Example 62. A system according to any one of examples 54 to 61, comprising a supervisor interface configured to deliver a human detectable indication to a supervisor of said NF training, and wherein said control circuitry is configured to deliver a human detectable indication to said supervisor using said supervisor interface with information regarding a change in activity of said at least one mesolimbic brain region relative to a threshold value or a target activity level during said measure of said EEG signals and/or following said NF training.

Example 63. A system according to any one of examples 54 to 62, wherein said at least one algorithm comprises a model correlating EEG signals recorded from a specific set of electrodes having a specific frequency range or specific frequency ranges that were recorded during a specific time window, with fMRI-BOLD activity of said at least one mesolimbic brain region, and wherein said control circuitry is configured to use said model to identify in said measured EEG signals, a subset of EEG signals indicating an activity of said at least one mesolimbic brain region, and to detect said increase in activity based on said identified subset of EEG signals.

Example 64. A system for delivery of a neurofeedback (NF) training, comprising:
a trainee interface configured to deliver at least one human detectable indication to a subject;
a control unit, comprising:
a memory;
an EEG recording unit, configured to receive electrical signals from one or more electrodes positioned on a head of a subject;
a control circuitry, wherein said control circuitry is configured to:
signal said trainee interface to deliver a sensory indication stored in said memory to a subject, wherein said sensory indication is selected to increase a pleasure response in said subject brain;
measure EEG signals according to the electrical signals received by said EEG recording unit;
detect an activity level of at least one mesolimbic brain region relative to a target activity level, based on said measured EEG signals and using at least one algorithm stored in said memory, wherein said detected activity level indicates a level of said pleasure response in said subject;
lower said target activity level if said detected activity level is lower than said target activity level;
and
repeat said signal said measure and said detect using said lowered target activity level.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as determining an activity level of a brain region, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a scheme of different components of the positive valence system;

FIG. 1B is a flow chart of a general process for training a selected subject or a selected population of subjects, according to some exemplary embodiments of the invention;

FIG. 1C is a flow chart of a general process for treating subjects diagnosed with Anhedonia, according to some exemplary embodiments of the invention;

FIGS. 1D, 1E and 1F are graphs showing a change in a pleasure response of a subject over time, according to some exemplary embodiments of the invention;

FIG. 1G is graph showing a change in feedback over time, according to some exemplary embodiments of the invention;

FIG. 2A is a flow chart of a treatment program, according to some exemplary embodiments of the invention;

FIG. 2B is a flow chart of a treatment session, according to some exemplary embodiments of the invention;

FIG. 2C is a flow chart of a NF cycle, according to some exemplary embodiments of the invention;

FIG. 3 is a block diagram of a system for delivery of the treatment, according to some exemplary embodiments of the invention;

FIG. 4 is a flow chart describing the system activities, according to some exemplary embodiments of the invention;

FIGS. 5A and 5B are flow charts describing the system activities divided into NF session blocks, according to some exemplary embodiments of the invention;

FIGS. 6A-6F are images of a patient visual interface presenting a scheme of the brain, according to some exemplary embodiments of the invention;

FIGS. 7A-7D are images of a different patient visual interface, according to some exemplary embodiments of the invention;

FIGS. 8A and 8B are graphs presented in a supervisor interface of the system, according to some exemplary embodiments of the invention; and

FIGS. 9A-9C are graphs showing a change in scores in a SHAPS-C assessment scale (9A), a HDRS assessment scale (9B), and a CGI-I assessment scale measured during an experiment in a group of MDD patients performing the NF treatment.

### DESCRIPTION OF SPECIFIC EMBODIMENTS OF THE INVENTION

The present invention, in some embodiments thereof, relates to modulating an activity of at least one mesolimbic brain region and, more particularly, but not exclusively, to modulating an activity of at least one mesolimbic brain region related to a reward system in a brain.

### Overview

A broad aspect of some embodiments relates to delivering a neurofeedback (NF) training, also termed herein as a NF treatment, in order to treat deficiencies in the positive valence system and/or to enhance activity of the positive valence system. In some embodiments, the NF training is delivered to subjects diagnosed with depression, for example with Major Depressive Disorder (MDD) and/or Anhedonia. Alternatively or additionally, the NF training or parts thereof is used to treat subjects diagnosed with Substance Use Disorder (SUD).

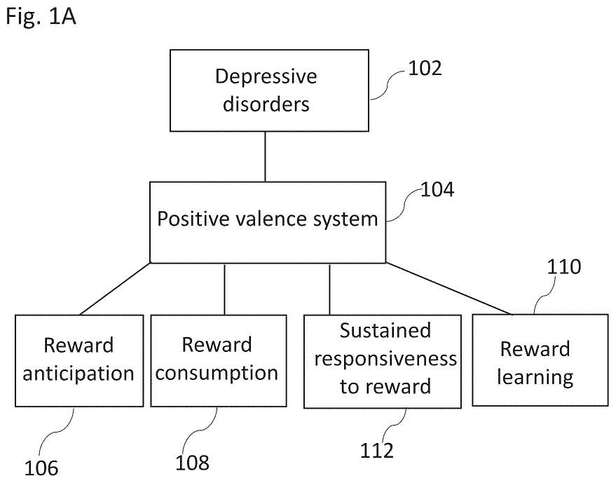

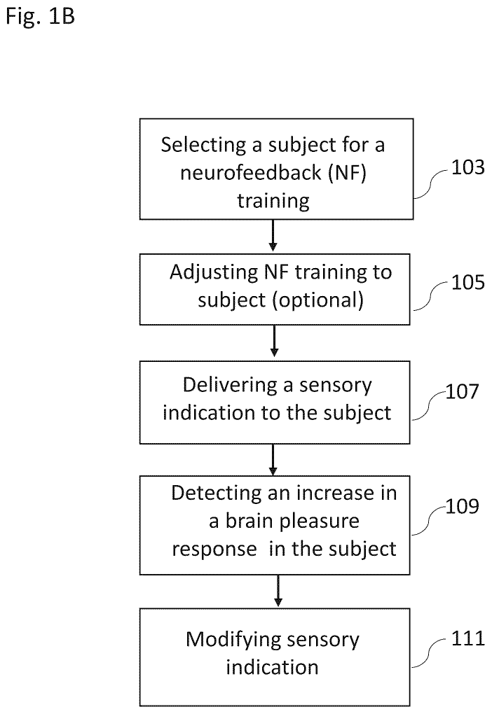

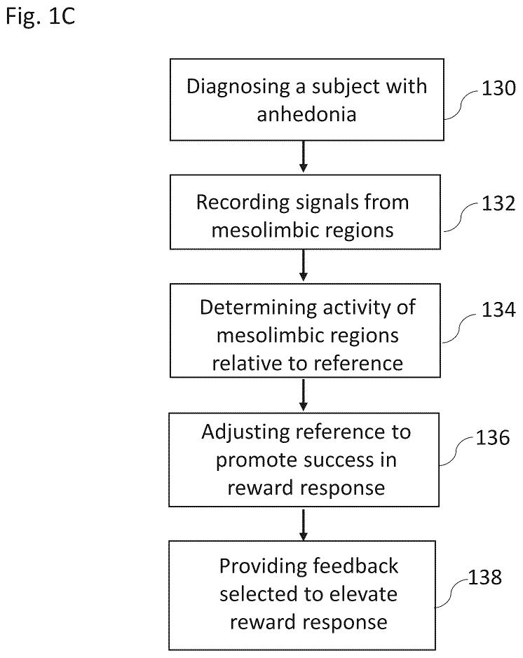

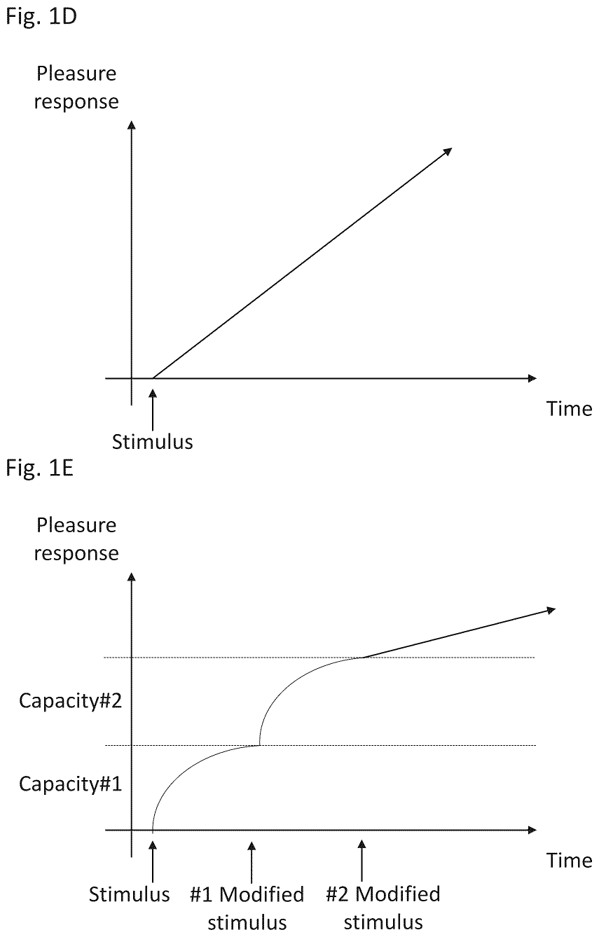

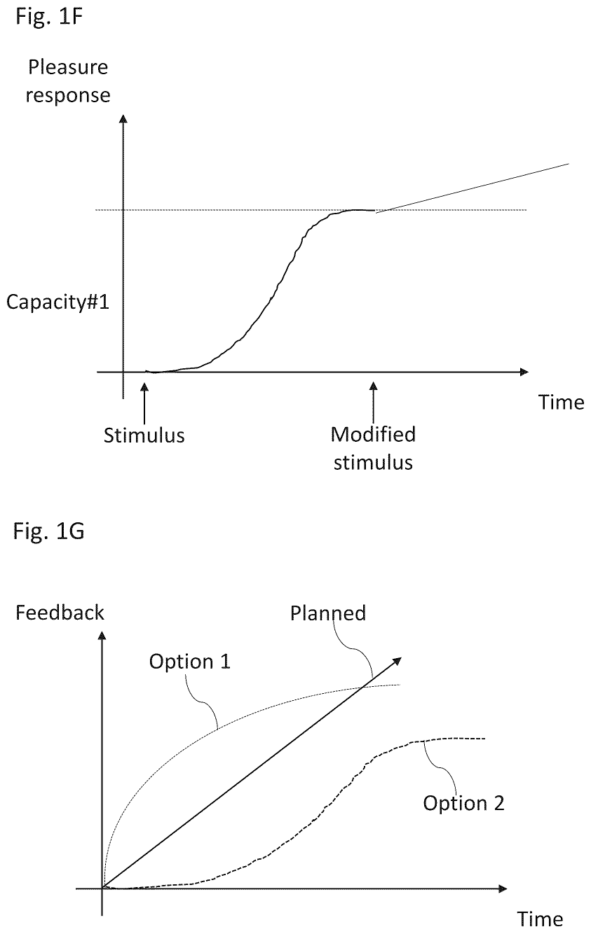

Studies of mental health disorders identified two systems that underlie anxiety and depressive disorders: the positive and negative valence systems. The negative valence system relates to threat sensitivity, for example how responsive one is to immediate, distal, or sustained threat, loss, and removal of reward (Kozak & Cuthbert, 2016). As shown in FIG. 1A, the positive valence system 104 is related to responsivity to reward (Kozak & Cuthbert, 2016). More specifically, the positive valence system 104 can be broken down into anticipation 106, consumption 108, and learning of reward 110. That is, one can experience deficits or elevations in anticipating or expecting future positive outcomes (anticipation of reward 106), in recognizing or appreciating positive experiences (consumption of reward 108), and in learning how to obtain future rewards from prior reward obtainment (learning of reward 110). Reward is defined as a positive, pleasurable, or desirable experience, outcome, or object.

Another component of the positive valence system that is altered in MDD patients is sustained responses to rewards 112. Aberrant sustained responses to rewards have been proposed as a core contributor to depression symptoms, particularly anhedonia (Tomarken & Keener, 1998).

In clinical research, anhedonia is a term used to describe a deficit or dysfunction of any part of the positive valence system (Thomsen, Whybrow, & Kringelbach, 2015). Traditionally, however, clinical practitioners have used this word to mean a loss of interest or pleasure, which is a core symptom of depression (Thomsen et al., 2015). It continues to be used as a major diagnostic criterion in the most recent Diagnostic and Statistical Manual of Mental Disorders, Fifth Edition (DSM-5; American Psychiatric Association, 2013), the clinical handbook utilized by practitioners in the United States. DSM-5 (American Psychiatric Association, 2013) defines major depressive disorder as (a) a 2-week period of low mood or loss of interest or pleasure in daily activities (i.e., anhedonia) that represents a change from baseline and results in significant functional impairment and (b) the presence of three or four additional symptoms (i.e., changes in appetite, sleep problems, psychomotor agitation/retardation, loss of energy, poor concentration, thoughts of worthlessness, suicidal ideation) (Dour et al. 2019 “Psychological Treatments for Anhedonia: Reward Anticipation, Consumption, and Learning”).

According to some exemplary embodiments, the NF treatment, also termed herein as neurofeedback training is a functional magnetic resonance imaging (fMRI) neurodeeback. Alternatively, the NF treatment is delivered by recoding EEG signals from a subject brain, for example without using functional magnetic resonance imaging during the delivery of the NF treatment.

An aspect of some embodiments relates to training at least one selected subject or a population of subjects, to increase an activity of a brain positive valence system. In some embodiments, the at least one selected subject or population of subject is expected to experience at least one symptom of depression in the future, for example at least 1 day, at least 1 week, at least one month, at least one year, or any intermediate, shorter or longer time period, from ending the training. In some embodiments, the at least one selected subject or population of subjects are trained to increase the activity of a brain positive valence system by increasing a pleasure response in the brain.

According to some embodiments, the training is a neurofeedback (NF) training which comprises presenting to a trainee a sensory indication, and instructing the trainee to modify the presented sensory indication, optionally by performing at least one specific activity. In some embodiments, the sensory indication is selected to increase a pleasure response in the brain of the trainee and/or is an indicator of the brain pleasure response in the trainee. In some embodiments, the sensory indication comprises a visual and/or an audio indication delivered to the trainee. Optionally, the sensory indication is a stimulus.

According to some embodiments, the NF training further comprises detecting an increase in a brain pleasure response, optionally following the instructing, and providing a feedback signal, for example a human detectable indication, to the trainee indicating the increase in the brain pleasure response. In some embodiments, the feedback signal is provided to the trainee during the presenting. In some embodiments, the feedback signal is provided by modifying the sensory indication presented to the trainee. In some embodiments, the feedback signal is provided to the trainee by modifying the sensory indication in a way selected to increase the brain pleasure response in the subject, for example by modifying the sensory indication to include additional pleasant visual and/or audio indications or ques.

According to some embodiments, the NF training is a tool for teaching a subject how to increase a pleasure response in his own brain by performing a specific activity, for example a specific mental or cognitive task that is optionally personalized for him. The subject will be able to apply this specific mental or cognitive task in the future when experiencing or encountering at least one symptom of depression.

According to some embodiments, the NF training described in the application is delivered to a predetermined selected population of subjects expected to experience at least one symptom of depression in the future. In some embodiments, the NF training is delivered using the system and/or device described in the application. In some embodiments, the at least one symptom is a symptom of depression according to the Diagnostic and Statistical Manual of Mental Disorders, Fifth Edition (DSM-5; American Psychiatric Association, 2013).

According to some embodiments, the at least one symptom comprises depressed mood and/or anhedonia. Additionally or alternatively, the at least one symptom of depression comprises at least one of, weight loss, weight gain, insomnia, hypersomnia, psychomotor agitation, psychomotor retardation, reduction in energy, worthlessness, guilt, concentration, suicidal thoughts and/or suicidal behaviors.

According to some embodiments, the predetermined selected population comprise subjects having a relative, for example a first degree relative, previously diagnosed with depression or major depression. In some embodiments, the predetermined selected population comprise subjects that are first responders, for example a person with specialized training who is among the first to arrive and provide assistance or incident resolution at the scene of an emergency, such as an accident, disaster, medical emergency, fire, crime, or terrorist attack.

According to some embodiments, the predetermined selected population comprise subjects diagnosed with at least one of, post-traumatic stress disorder (PTSD), attention deficit hyperactivity disorder (ADHD), substance-induced mood disorder, chronic kidney disease (CKD) and/or Premenstrual dysphoric disorder (PMDD). In some embodiments, the predetermined selected population comprise subjects in a risk for developing peripartum depression or subjects already diagnosed or previously diagnosed with peripartum depression, for example a male or a female subject diagnosed with peripartum depression in a previous pregnancy.

According to some embodiments, the predetermined selected population comprise subjects in a risk for developing seasonal affective disorder (SAD) or that were previously diagnosed with SAD or experienced SAD.

According to some embodiments, the predetermined selected population comprise subjects diagnosed with cancer, for example pancreatic cancer or lung cancer.

According to some embodiments, the predetermined selected population comprise subjects diagnosed with Chronic Kidney Disease (CKD)

An aspect of some embodiments related to delivering of a stimulus to a subject that is selected to increase a reward response in the subject brain. In some embodiments, the stimulus is modified in a way that is selected to further increase the reward response in the subject. In some embodiments, the delivered stimulus increases the reward response by increasing a pleasure response in the subject brain, for example increasing a release of dopamine in one or more brain regions of the reward system. In some embodiments, the stimulus is optionally delivered via a sensory indication, for example a patient interface that includes an interface with different scenarios, audio, visual, tactile or any sensory human detectable indication.

According to some embodiments, an increase in a pleasure response is detected in the subject, following and/or during the delivery of the response. In some embodiments, the increase in pleasure response is detected by detecting modulation in activity of at least one brain region, for example a mesolimbic brain region.

According to some embodiments, detecting modulation in activity and/or determining an activity of the at least one brain region is based on recording of electrical signals from at least one electrode, for example at least one electrode of array of electrodes positioned on a head of a subject. In some embodiments, the at least one electrode or array of electrodes are configured to measure EEG signals. Alternatively, the electrical signals are recorded directly from at least one brain region using an implanted electrode. In some embodiments, an electrode implanted in at least one mesolimbic region, for example the VS allows recording of electrical signals, for example as described in Delaloye S. et. al., 2014 “deep brain stimulation in the treatment of depression”.

Alternatively or additionally, the increase in pleasure response is detected by detecting a physiological response of the subject indicating a pleasure response, for example by detecting of a smile or a change in the face of the subject, optionally using an optic sensor or a camera. Alternatively or additionally, the pleasure response is detected using at least one sensor that detects changes in at least one of, skin conduction, heart rate, and muscle functioning.

According to some embodiments, modulation in activity and/or determining an activity of the at least one brain region is performed based on EEG signals and using at lets one electrical fingerprint (EFP) of at least one brain region, for example at least one mesolimbic brain region. In some embodiments the EFP is based on EEG signals that correlate with fMRI-BOLD activity of the at least one mesolimbic brain region, for example the VS.

According to some embodiments, the EFP is a model linking EEG measurements to a fMRI-BOLD signal indicating a selective activation of at least one specific brain region, for example the VS. In some embodiments, the model comprises a coefficient matrix of at least 10 coefficients corresponding to frequency bands, electrodes and one or more time windows. In some embodiments, activity of at least one brain region or a modulation in the activity of at least one brain region is performed, for example as described in WO2021260697A1 or in WO2012104853A2, incorporated herein as a reference in their entirety.

According to some embodiments, the EFP model is applied on EEG signals recorded from the at least one electrode or from an array of electrodes. In some embodiments, changes in the EFP indicate changes in activity of the at least one brain region.

According to some embodiments, a method for neurofeedback assessment, includes selecting a population of subjects currently experiencing or expected to experience in the future, at least one symptom of depression, for example as described above. In some embodiments, the method further includes presenting optionally using a user interface of a system, to each subject of the selected population of subjects, the sensory indication configured to indicate or induce a pleasure response in the subject brain, and instructing the subject optionally using the user interface, to modify the presented sensory indication by performing at least one specific activity, for example a cognitive activity or a mental activity. In some embodiments, the method further includes measuring optionally by a control circuitry, electrical signals from the subject brain, detecting optionally by the control circuitry, an increase in the brain pleasure response in the subject based on the measured electrical signals. In some embodiments, a feedback signal indicating the increase in the brain pleasure response is generated and delivered to the subject while presenting the sensory indication, optionally by said user interface, for example as described above.

An aspect of some embodiments relates to providing a suppressing signal to a subject, for example to downregulate an activity level of at least one brain region. In some embodiments, the suppressing signal is delivered when an increase in activity of at least one mesolimbic brain region is detected, in a subject diagnosed with Mania.

According to some embodiments, the treatments and methods described herein is used for conditioning of the patient to allow, for example, practicing one or more of the strategies which may involve one or more of the patient interfaces, while the patient is at his home or outside the clinic.

According to some embodiments, the system described herein may be part of an overall treatment, in which the activity of the VS striatum is modulated, while modulating at least one different brain region, either using a different or similar NF treatment or using at least one drug.

An aspect of some embodiments relates to performing the NF training before and/or after administering at least one medicine, for example a drug or a drug compound for treating depression. In some embodiments, the drug is administered according to a known administration regimen when treating depression before and/or after performing the NF training, for example before and/or after performing at least one training session (treatment session). Alternatively, the administration regimen of the drug, for example the drug dosage, is modified, for example reduced, when administering the drug as part of a treatment protocol which includes administration of the drug and performance of the NF training. In some embodiments, the administration regimen of the drug, for example the drug dosage, is modified based on a progress of the subject in modulating the activity level of the at least one brain region or brain network as described in this application during the NF training. Optionally, the administration regime of the subject is modified based on results of an assessment of the subject mental and/or cognitive state performed during a NF training session, when finishing a NF training session, between training sessions, or after finishing the NF treatment. In some embodiments, the timing for performing the NF training is adjusted according to the specific drug, for example according to at least one of pharmacokinetics and pharmacodynamics of the drug. Alternatively or additionally, the timing for performing the NF training is adjusted according to the effect of the drug on the trainee, for example to make sure the trainee mental and/or cognitive state is suitable for performing the NF training.

According to some embodiments, the drug belongs to a family of drugs, for example Selective Serotonin Reuptake Inhibitors (SSRI), Serotonin and Norepinephrine Reuptake Inhibitors (SNRI), Tricyclic and Tetracyclic Antidepressants, Atypical Antidepressants, and Monoamine Oxidase Inhibitors (MAOIs). Alternatively, the drug is an N-methyl D-aspartate (NMDA) Antagonist, for example esketamine, or a Neuroactive Steroid Gamma-Aminobutyric Acid (GABA)-A Receptor Positive Modulator, for example brexanolone.

According to some embodiments, the NF training comprises presenting to a trainee, for example the subject taking the medicine, a sensory indication, and instructing the trainee to modify the presented sensory indication, optionally by performing at least one specific activity. In some embodiments, the sensory indication is selected to increase a pleasure response in the brain of the trainee. In some embodiments, the sensory indication comprises a visual and/or an audio indication delivered to the trainee. Optionally, the sensory indication is a stimulus.

According to some embodiments, the NF training further comprises detecting an increase in a brain pleasure response, optionally following the instructing, and providing a feedback signal, for example a human detectable indication, to the trainee indicating the increase in the brain pleasure response. In some embodiments, the feedback signal is provided to the trainee during the presenting. In some embodiments, the feedback signal is provided by modifying the sensory indication presented to the trainee. In some embodiments, the feedback signal is provided to the trainee by modifying the sensory indication in a way selected to increase the brain pleasure response in the subject, for example by modifying the sensory indication to include additional pleasant visual and/or audio indications or ques.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Training

According to some exemplary embodiments, the NF protocol is delivered as a NF treatment for training subjects currently suffering from one or more symptoms of depression or that are diagnosed with MDD, how to increase an activity of their positive valence system, for example how to increase a pleasure response in their own brain by performing at least one activity. Alternatively or additionally, the NF protocol is delivered as a NF training to subjects, for example subjects that are currently not experiencing a symptom of depression, that are expected to experience a symptom of depression in the future. In some embodiments, the NF training teaches the trainees how to cope with a symptom of depression in the future, for example by performing the at least one activity to increase the pleasure response in their brain.

Reference is now made to FIG. 1B, depicting a general NF training, according to some exemplary embodiments of the invention.

According to some exemplary embodiments, a subject is optionally selected for the NF training, at block 103. In some embodiments, in some embodiments, the subject is part of a predefined population of subjects selected for the NF training. In some embodiments, the selected subject is a subject expected to experience at least one symptom of depression in the future. In some embodiments, the at least one symptom comprises a symptom of depression according to the Diagnostic and Statistical Manual of Mental Disorders, Fifth Edition (DSM-5; American Psychiatric Association, 2013). In some embodiments, the at least one symptom comprises depressed mood and/or anhedonia. Additionally or alternatively, the at least one symptom of depression comprises at least one of, weight loss, weight gain, insomnia, hypersomnia, psychomotor agitation, psychomotor retardation, reduction in energy, worthlessness, guilt, concentration, suicidal thoughts and/or suicidal behaviors.

According to some exemplary embodiments, the selected subject is a subject diagnosed with at least one of, post-traumatic stress disorder (PTSD), attention deficit hyperactivity disorder (ADHD), substance-induced mood disorder, chronic kidney disease (CKD) and/or Premenstrual dysphoric disorder (PMDD). In some embodiments, the selected subject is a subject in a risk for developing peripartum depression or is already diagnosed or previously diagnosed with peripartum depression, for example a male or a female subject diagnosed with peripartum depression in a previous pregnancy.

In some embodiments, the selected subject is a subject in a risk for developing seasonal affective disorder (SAD) or a subject previously diagnosed with SAD or previously experienced SAD. In some embodiments, the selected subject is a subject diagnosed with cancer, for example pancreatic cancer or lung cancer.

According to some exemplary embodiments, the selected subject is a subject having a relative, for example a first degree relative, previously diagnosed with depression or major depression. In some embodiments, the selected subject is a subject that is a first responder, for example a person with specialized training who is among the first to arrive and provide assistance or incident resolution at the scene of an emergency, such as an accident, disaster, medical emergency, fire, crime, or terrorist attack.

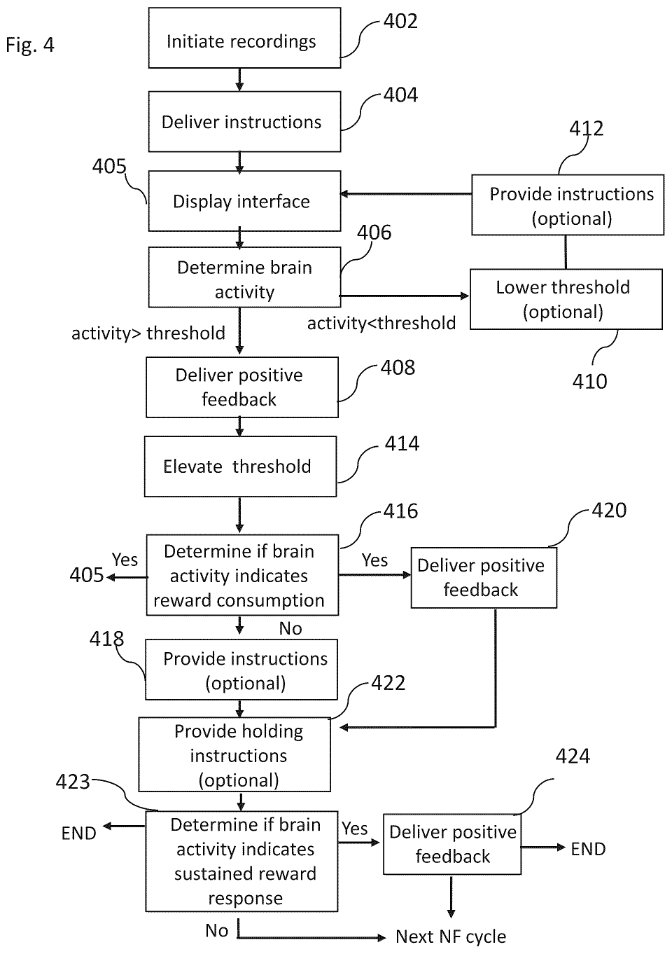

According to some exemplary embodiments, the NF training is optionally adjusted according to characteristics of the selected subject, at block 105. In some embodiments, the overall length of the NF training is reduced or increased. In some embodiments, at least one sensory indication included in the NF training is adjusted or replaced according to characteristics of the selected subject. In some embodiments, the number of training sessions and/or a length of each or at least one training session is adjusted according to characteristics of the selected subject. In some embodiments, the NF training is delivered to the trainee using the system 302 shown in FIG. 3.

According to some exemplary embodiments, during the NF training, a sensory indication is delivered, for example presented to the selected subject at block 107. In some embodiments, the sensory indication comprises a visual and/or an audio indication. In some embodiments, the sensory indication, optionally a stimulus, is configured to evoke or induce a pleasure response in the subject brain. In some embodiments, the sensory indication is delivered using an interface, for example a trainee interface also termed herein as a patient interface, for example interface 306 shown in FIG. 3.

According to some exemplary embodiments, an increase in a brain pleasure response in the subject brain is detected at block 109. In some embodiments, the increase in the brain pleasure response is detected based using electrical signals recorded from the subject brain, for example, using the electrodes 316 and 318 shown in FIG. 3. In some embodiments, the recorded electrical signals are processed and are used for measuring electroencephalogram (EEG) signals, which indicate an increase in activity of at least one mesolimbic brain region in response to delivered sensory indication.

According to some exemplary embodiments, the sensory indication presented to the subject is modified at block 111. In some embodiments, the sensory indication is modified based on the detected increase in the brain pleasure response. In some embodiments, the sensory indication is modified in a way configured or designed to increase the brain pleasure response in the subject, for example by adding pleasant visual and/or audio indications to the sensory indication or by delivering pleasant visual and/or audio indications to the subject in addition to the sensory indication.

According to some exemplary embodiments, the delivering of the sensory indication, the detecting of the increase in the brain pleasure response and the modifying of the sensory indication according to the detected increase, are repeated continuously for at least 1 minute, for at least 3 minutes, for at least 5 minutes or any intermediate, shorter or longer time period during a session of the NF training.

### Exemplary General Process for Anhedonia Treatment

As described above anhedonia is a term used to describe a deficit or dysfunction of any part of the positive valence system. Clinicians also use this word to mean a loss of interest or pleasure, which is a core symptom of depression. Reference is now made to FIG. 1C, depicting a general process for treating a subject diagnosed with anhedonia, according to some exemplary embodiments of the invention.

According to some exemplary embodiments, a subject is diagnosed with Anhedonia, at block 130. In some embodiments, the subject is diagnosed at block 130 with a dysfunction of the positive valence system, for example a dysfunction in at least one of, a dysfunction in reward anticipation, a dysfunction in reward consumption, and/or a dysfunction in sustained responsiveness to reward. In some embodiments, the subject is diagnosed with Anhedonia, optionally MDD with Anhedonia, using at least one of the Diagnostic and Statistical Manual of Mental Disorders, Fifth Edition (DSM5), and a score≥25 in the Snaith-Hamilton pleasure scale for clinicians (SHAPS-C). Optionally, MDD diagnosis is determined via the Neuropsychiatric Interview (MINI for DSM-5).

Optionally, information collected while the subject uses the system and/or performs the treatment protocol is used to improve the patient initial diagnosis, and/or is used as an independent diagnosis tool.

According to some exemplary embodiments, signals are recorded from at least one mesolimbic region of the brain, at block 132. In some embodiments, the signals are recorded from at least one brain region related to the reward system, for example the Ventral Striatum (VS), the Nucleus Accumbens, Ventrall tegmental area, OrbitoFrontal cortex, and the Insula. In some embodiments, the signals are recorded by one or more electrodes positioned on a head of a subject that are optionally used to measure electroencephalography (EEG) signals. In some embodiments, the one or more electrodes are positioned in at least one location on a head of a subject, of locations C3, C4, Cz, FCZ, P3, Pz and P4 of the extended 10-20 coordinate system. In some embodiments, 5 electrodes, each is positioned in one of the locations C3, C4, Cz, FCZ, P3, Pz and P4, are used

According to some exemplary embodiments, activity of at least one mesolimbic brain region is determined at block 134. In some embodiments, the activity of the at least one mesolimbic brain region is determined based on the recordings performed at block 132. In some embodiments, the activity of the at least one mesolimbic brain region is determined using at least one electrical fingerprint (EFP) of the at least one mesolimbic brain region, for example an EFP of the ventral striatum (VS). In some embodiments the EFP is based on EEG signals that correlate with fMRI-BOLD activity of the at least one mesolimbic brain region, for example the VS.

According to some exemplary embodiments, the EFP is a model linking EEG measurements to a fMRI-BOLD signal indicating a selective activation of at least one specific brain region, for example the VS. In some embodiments a selective activation of a brain region means activation of the at least one specific brain region in a level that is higher from activation levels of other brain regions, for example higher in more than 30% of the activity of other brain regions, for example higher in more than 50% of the activity of other brain regions, higher in more than 60% of the activity of other brain regions, higher in more than 80% of the activity of other brain regions, higher in more than 90% of the activity of other brain regions, or any higher or lower percentage value of the activity of other brain regions. Alternatively or additionally, a selective activation of at least one specific brain region means activation of the at least one specific brain region which is higher relative to a reference value optionally used as a baseline, or a value or indication thereof previously measured from the at least one specific brain region. In some other embodiments, the EFP is used to measure an activity at any given moment and overtime assess the regions accumulative activity.

According to some exemplary embodiments, the model comprises a coefficient matrix of at least 10 coefficients corresponding to frequency bands, electrodes and one or more time windows. In some embodiments, the EFP comprises electrical signals, for example EEG electrical signals recorded from EEG electrodes located at one or more positions on a head of a subject, for example positions C3, C4, Cz, FCZ, P3, Pz and P4. Alternatively, the EFP comprises electrical signals, for example EEG electrical signals recorded from EEG electrodes located at one or more positions C4, F7, F8, T7, T8, P8, TP9 and TP10. In some embodiments, the EFP comprises EEG electrical signals in a frequency range between 0-1 GHZ, and in a time delay window between 0 and 30 seconds, for example in a time delay window between 0 and 10 seconds, in a time delay window between 0 and 15 seconds, in a time delay window between 0 and 20 seconds, or any intermediate, smaller or larger time delay window.

According to some exemplary embodiments, the activity of the at least one mesolimbic brain region is determined at block 134 using an EFP of the VS, for example as described in WO2021260697A1, incorporated herein as a reference in its entirety.

According to some exemplary embodiments, the activity of the at least one mesolimbic brain region is determined at block 134 relative to a reference value. In some embodiments, the reference value indicates a threshold for a target activation level, for example a desired activation level, of the at least one mesolimbic brain region, for example the VS. In some embodiments, the reference value is determined based on clinical assessment of the subject, for example clinical assessment performed during the diagnosis at block 130. Alternatively or additionally, the reference value is determined based on performance of the subject and/or information collected from the subject during the NF treatment. Alternatively or additionally, the reference value is determined based on clinical assessment and/or performance of one or more different subjects in the NF treatments and/or information collected from the one or more different subjects before, during and/or after a NF treatment.

According to some exemplary embodiments, the activity of the at least one mesolimbic brain region is determined while the subject performs a cognitive task. In some embodiments, performing the cognitive task modulates the activation level of the at least one mesolimbic brain region.

According to some exemplary embodiments, the reference value is adjusted to promote success in a reward response in the subject, at block 136. In some embodiments, a reward response is any reward system response related to reward anticipation, reward consumption, and/or reward holding, for example reward sustained response. In some embodiments, if the activity of the at least one mesolimbic brain region is lower than the target reference value, then the reference value is lowered, at block 136. In some embodiments, the lowering degree of the reference value changes between subjects and/or changes according to the performance of the subject during the NF treatment. Alternatively, the lowering degree of the reference value is fixed, or is predetermined.

According to some exemplary embodiments, a feedback signal is provided to the subject at block 138. In some embodiments, the feedback signal is selected to elevate a reward response, for example a reward anticipation response in the subject. In some embodiments, the feedback signal is provided when the determined activity of the at least one mesolimbic brain region is higher than the reference value.

Optionally, the system provides the patient a reward even if the determined activity is lower than the threshold, for example to provide a false feeling of success.

According to some exemplary embodiments, the feedback signal comprises a visual and/or an audio signal and/or tactile and/or olfactory signal. In some embodiments, the feedback signal is provided by modifying a visual interface presented to the subject. In some embodiments, the visual interface provides a visual feedback, optionally a continuous visual feedback, indicating an activity level of the at least one mesolimbic brain region. In some embodiments, the visual interface is adjusted in a delay window of between 5-20 seconds, for example 5-15 seconds, 10-20 seconds, 10-15 seconds or any intermediate, smaller or larger delay window, relative to signals recorded at block 132, to indicate an activity level of the at least one mesolimbic brain region.

### Exemplary Change in Pleasure Response

According to some exemplary embodiments, the NF method and system described in this application provide stimulus to a subject, for example as an interface or a patient interface, which is selected to increase a reward response in the subject, for example by increasing a pleasure response of the subject to the provided stimulus.

Reference is now made to FIGS. 1D-IF depicting a change in a pleasure response of a subject in response to a stimulus, according to some exemplary embodiments of the invention.

According to some exemplary embodiments, for example as shown in FIG. 1D, a change in a pleasure response following an exposure to a stimulus is linear. In some embodiments, a linear phase of the change in the pleasure response is limited for a specific time period.

According to some exemplary embodiments, for example as shown in FIGS. 1E and 1F, a pleasure response of a subject, reaches a plateau following a stimulus. In some embodiments, the plateau indicates a limited capacity of the subject to increase a pleasure response in response to the stimulus. In some embodiments, the NF system, for example the system described in FIG. 3, modifies the stimulus in a way that increases a capacity of the subject to response to a stimulus in a way that increases a pleasure response. In some embodiments, for example as shown in FIG. 1E, the system further modifies the stimulus in order to increase the capacity of the subject to increase a pleasure response. In some embodiments, the system modifies the stimulus in order to reach a predetermined and/or a desired, for example a target level of pleasure response.

According to some exemplary embodiments, for example as shown in FIG. 1G, the system is adjusted to deliver a feedback, for example a stimulus, in a specific pattern that changes over time. In some embodiments, for example as shown in FIG. 1G, the system is configured to deliver a planned feedback that linearly increases over time. In some embodiments, during the treatment, the system modifies the feedback and/or applies feedback having different optional patterns, for example when the change in the pleasure response of the subject is not linear and/or when the change in the pleasure response reaches a plateau. In some embodiments, the different feedback pattern are stored in a memory of the system. In some embodiments, the system determines which pattern to use, using at least one algorithm or a look-up table stored in the memory of the system.

### Exemplary Treatment Protocol

According to some exemplary embodiments, the NF treatment is used to treat subjects diagnosed with at least one of, MDD, MDD with Anhedonia, Anhedonia, and subjects diagnosed with deficiencies in the rewards system or deficiencies in the positive valence system, for example deficiencies in reward anticipation, deficiencies in reward consumption, deficiencies in sustained responsiveness to reward, and deficiencies in reward learning. In some embodiments, the treatment is performed in a supervisor clinic, for example in a clinic or office of a physician, a therapist or a social worker. Alternatively, or additionally, at least part of the treatment is performed in the house or home of the subject. In some embodiments, at least part of the treatment is performed without a presence of a supervisor during the training, for example when performing at least one transfer cycle in which feedback is not provided to the trainee.

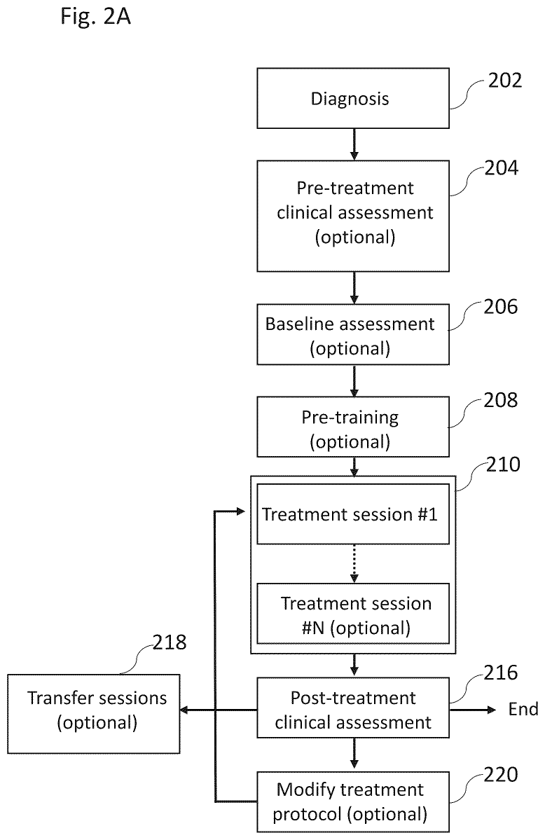

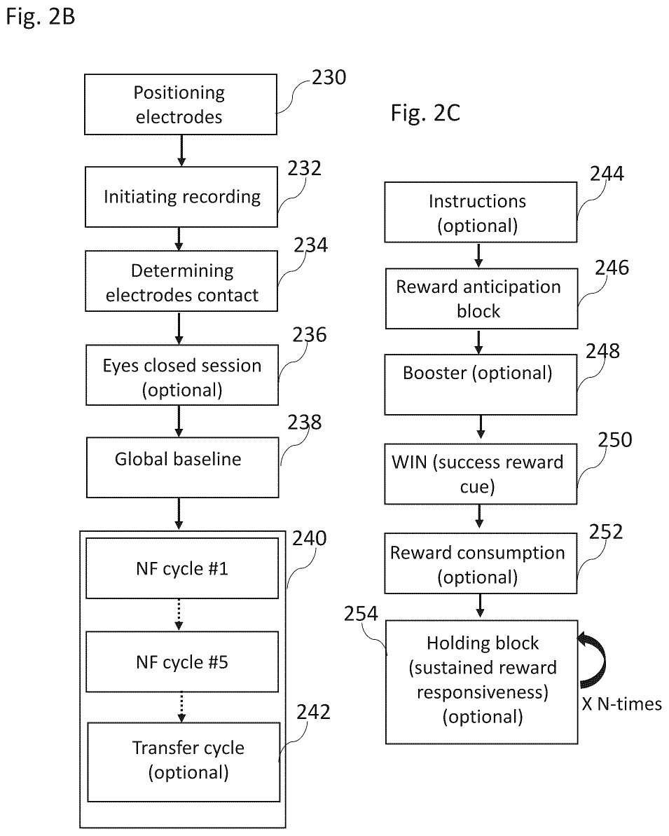

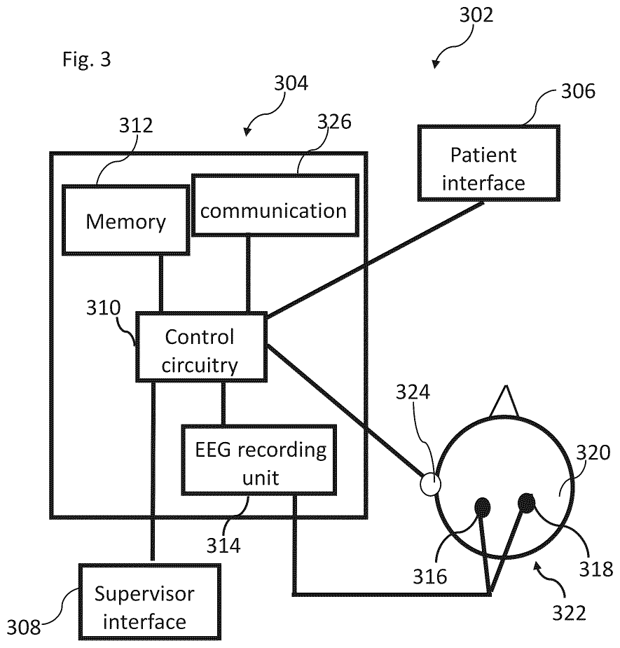

Reference is now made to FIG. 2A, depicting a treatment program, for example a NF treatment program, according to some exemplary embodiments of the invention.

According to some exemplary embodiments, a subject, for example a male or a female subject, is diagnosed at block 202. In some embodiments, the subject is diagnosed with at least one of MDD, MDD with Anhedonia, Anhedonia, reward system deficiencies, and/or deficiencies in the positive valence system. In some embodiments, the subject is diagnosed as previously described at block 130. Alternatively or additionally, the subject is diagnosed according to the Diagnostic and Statistical Manual of Mental Disorders (DSM) of the American Psychiatric Association, for example the fifth edition of the DSM (DSM-5).

According to some exemplary embodiments, the subject is diagnosed using one or more questionnaires, interviews, observation sessions, imaging analysis techniques, for example computed tomography (CT), Magnetic resonance imaging (MRI), Positron emission tomography (PET), or one or more biomarkers. In some embodiments, the subject is diagnosed by a physician or a psychologist who is an expert in mental health. In some embodiments, the metal health experts decides whether the subject may benefit from the NF treatment or from a different treatment, for example pharmacotherapy using a drug, or a combination of both a NF treatment and pharmacotherapy. In some embodiments, if the mental health expert decides that the diagnosed subject is a good candidate for the NF treatment, he refers the subject to an expert who performs the NF treatment, for example a therapist.

According to some exemplary embodiments, a pre-treatment clinical assessment is performed at block 204. In some embodiments, the clinical assessment is performed by the expert who is optionally supervising the NF treatment. In some embodiments, the pre-treatment clinical assessment is performed in order to make sure that the diagnosed subject is suitable for the NF treatment. In some embodiments, the pre-treatment clinical assessment uses one or more of the diagnostic tools described at block 202. Alternatively or additionally, during the clinical assessment, the expert examines whether the diagnosed subject, for example a patient, is capable of performing the NF treatment using the treatment system, and/or the response of the subject to the patient interface of the system. In some embodiments, the clinical assessment is performed at a clinic and/or at home.

According to some exemplary embodiments, during the clinical assessment performed at block 204, one or more parameters of the NF treatments are selected or adjusted for the specific patient. In some embodiments, the one or more parameters comprise at least one of, number of treatment sessions, duration of each treatment session, interval between two consecutive treatment sessions, number of NF cycles in each treatment session, and type of patient interface.

Optionally, a baseline assessment is performed at block 206, for example to determine an initial clinical state of the subject prior to initiating the treatment. Optionally, during the baseline assessment, an initial threshold for an initial target activation level of the at least one mesolimbic brain region is determined. In some embodiments, the baseline assessment is performed using a calibration system in which the interaction between the subject and a patient interface is examined, optionally while monitoring the activity of the at least one mesolimbic brain region. Alternatively or additionally, the baseline assessment is performed during the pre-treatment clinical assessment, at block 204.

Optionally, a pre-training session is performed at block 208. In some embodiments, the pre-training session is performed by describing the flow of the treatment program to the patient and/or by practicing one or more exemplary treatment sessions or one or more NF cycles. In some embodiments, during the pre-treatment, the patient receives instructions or guidelines regarding application of one or more strategies, for example cognitive tasks, that may allow him to modulate the activation level of the at least one mesolimbic brain region. Alternatively or additionally, the patient receives instructions or guidelines to identify a preferred strategy, and/or how to identify the preferred strategy.

In some embodiments, instructions during the treatment process may be provided using at least one of audio, video, text, or verbally by the therapist. Alternatively, the subject does not receive instructions.

According to some exemplary embodiments, the treatment protocol includes one or more treatment sessions 210, for example training sessions, performed under the supervision of a supervisor, for example a therapist, a nurse, a social worker, or a psychologist. In some embodiments, the treatment sessions are performed in the clinic or office of the supervisor. In some embodiments, the treatment protocol includes up to 30 treatment sessions, for example up to 20 treatment sessions, up to 15 treatment sessions, up to 13 treatment sessions, up to 10 treatment sessions, or any intermediate, smaller or larger number of treatment sessions. In some embodiments, the treatment sessions are consecutive treatment sessions. In some embodiments, some of the sessions are provided at home or outside of the clinic.

According to some exemplary embodiments, each treatment session lasts between 5-60 minutes, for example 5-30 minutes, 10-30 minutes, 15-40 minutes, 20-50 minutes, or any intermediate, shorter or longer time period. In some embodiments, a time interval between two consecutive treatment sessions is at least 1 hour, for example at least 6 hours, at least 12 hours, at least 1 day, at least 3 days, or any intermediate, smaller or larger time interval. In some embodiments, a time interval between consecutive treatment sessions 210, is in a range between 1 day and 14 days, for example 1-3 days, 1-5 days, 1-7 days, 3-7 days, 3-10 days or any intermediate, smaller or larger range of values. In some embodiments, the treatment protocol comprises up to 4 treatment sessions in a week, for example up to 3 treatment sessions, up to 2 treatment sessions, or a single treatment session, in a week.

According to some exemplary embodiments, the patient undergoes post-treatment clinical assessment, at block 216. In some embodiments, the post-treatment clinical assessment is performed in order to determine a clinical state of the subject following the treatment sessions 210. Optionally, the post-treatment clinical assessment is performed as described at blocks 204 and/or 206. In some embodiments, if the post-treatment clinical assessment revels that a predetermined target of the treatment was achieved, then the treatment protocol ends. Alternatively or additionally, if the post-treatment clinical assessment revels that a predetermined target of the treatment was achieved, the patient is instructed to perform one or more transfer sessions 218, for example transfer sessions without feedback. Optionally, the patient performs the one or more transfer session at his home or at the clinic, while applying at least one strategy, for example at least one cognitive task that allows modulation of the at least one mesolimbic brain region, in a desired direction, as revealed during the treatment sessions 210. In some embodiments, the patient performs the one or more transfer sessions using an application software installed on a mobile device, for example a cellular device or a computer. Optionally, the patient performs the one or more treatment sessions by accessing an application software installed on a remote device, for example a server or a cloud storage positioned outside the patient home.

According to some exemplary embodiments, if the post-treatment clinical assessment revels that a predetermined target of the treatment was not achieved, then the treatment protocol is optionally modified at block 220. In some embodiments, modification of a treatment protocol comprises changing the number and/or duration of treatment sessions, and/or an interval duration between consecutive treatment sessions. Alternatively or additionally, modifications of a treatment protocol comprises modifying at least one parameter of the NF sessions performed during each session, for example type of patient interface presented to the patient, a reference value used as a threshold for determining a target, for example a desired activation level of the at least one mesolimbic brain region and/or any parameter related to the algorithms used for determining the activity of the at least one mesolimbic brain region and/or a relation between the determined activity and the reference value.

Optionally, after modifying the treatment protocol, the patient repeats one or more of the treatment sessions 210 according to the modified treatment protocol parameters.

### Exemplary Treatment Session

Reference is now made to FIG. 2B depicting a treatment session of the one or more treatment sessions 210 shown in FIG. 2A, according to some exemplary embodiments of the invention.

According to some exemplary embodiments, one or more electrodes are positioned on a head of the patient, at block 230. In some embodiments, the one or more electrodes comprise a plurality of electrodes positioned at different locations on a head of the subject, for example at locations C3, C4, Cz, FCZ, P3, Pz and P4 of the 10-20 coordinate system, on a head of the subject. In some embodiments, the electrodes are attached to a head of the patient, for example to the skull, using gel.

According to some exemplary embodiments, recording from the electrodes is initiated at block 232. In some embodiments, the electrodes are used to record EEG signals. In some embodiments, recording is initiated at block 232 before or after electrodes positioning at block 230.

According to some exemplary embodiments, contacts of the electrodes with the head of the subject is determined at block 234. In some embodiments, the contact is determined based on the recording initiated at block 232. In some embodiments, contact of the electrodes with the head of the patient is presented to the supervisor, for example using an interface showing electrodes with proper contact and electrodes that are not properly contacting the head of the patient. In some embodiments, the system alerts the supervisor and/or the user of which electrodes are not properly contacted during treatment and optionally what action to take to remedy the problem. In some embodiments, recording is initiated at block 232 after determining electrodes contact at block 234.

According to some exemplary embodiments, a treatment session optionally includes an eyes closed session, at block 236. In some embodiments, during the eyes close session signals are recorded from the patient when the patient is in a rest mode, for example to calibrate the recording and/or an EEG measurement system.

According to some exemplary embodiments, a treatment session optionally includes a global baseline session, at block 238. In some embodiments, during the global baseline session, EEG signals are measured and are collected for a pre-determined time period necessary to determine an activity of the at least one mesolimbic brain region, for example using an EFP of the mesolimbic brain region. In some embodiments, the pre-determined time period is in a range of 5-30 seconds, for example 5-20 seconds, 5-15 seconds, 5-13 seconds, 5-10 seconds, or any intermediate, smaller or larger range of values.

Optionally, a local baseline is calculated before every treatment session and/or before every NF cycle. In some embodiments, during the local baseline session, EEG signals are measured and are collected for a pre-determined time period necessary to determine an activity of the at least one mesolimbic brain region, for example using an EFP of the mesolimbic brain region. In some embodiments, the pre-determined time period is in a range of 5-30 seconds, for example 5-20 seconds, 5-15 seconds, 5-13 seconds, 5-10 seconds, or any intermediate, smaller or larger range of values.

According to some exemplary embodiments, a treatment session comprises one or more training cycles, at block 240. In some embodiments, the one or more training cycles comprise 1, 2, 3, 4, 5, 6, 7 or any larger number of NF training cycles. In some embodiments, the training cycles, for example the NF training cycles are consecutive training cycles. Optionally, the NF training cycles are continuously performed at block 240. Optionally, a delay between two consecutive NF training cycles is shorter than 30 seconds, for example shorter than 10 seconds, shorter than 5 seconds, shorter than 1 second or any intermediate, shorter or longer time period. Optionally, the training cycles comprise at least one transfer cycle 242, in which feedback regarding the activation level of the at least one mesolimbic brain region was not delivered to the patient.

According to some exemplary embodiments, during the NF cycles, the patient watches a patient interface, for example a visual, an audio, a tactile and/or an olfactory interface which presents a scenario that changes according to the determined activity level of the at least one mesolimbic brain region, while performing a task, for example a cognitive task. In some embodiments, the cognitive task is selected to increase the activity level of the at least one mesolimbic brain region. In some embodiments, an increase of the activity of the at least one mesolimbic brain region, increases or decreases the amount of stimulating cues delivered to the patient as part of the interface. Additionally, human detectable indications, for example visual, audio, tactile, and/or olfactory indications delivered to the subject while watching the interface indicate if a target activity level of the mesolimbic brain region was crossed or if the determined activity is closer or is in a desired direction towards the target activity level.

### Exemplary NF Cycle

Reference is now made to FIG. 2C depicting a NF cycle process, according to some exemplary embodiments of the invention.

According to some exemplary embodiments, instructions are optionally delivered at block 244 to the patient. In some embodiments, the instructions are delivered before a first NF cycle of a series of NF cycles that are performed during a single treatment session. In some embodiments, the instructions include a description of the following steps and/or a request from the patient to perform at least one task, for example a cognitive task to increase amount of stimulating cues delivered to the patient as part of the visual interface. In some embodiments, the instructions include a description of one or more specific tasks. Alternatively, the instructions include a request from the patient to try and find a task that will allow him to increase the amount of stimulating cues in the visual interface. In some embodiments, no instructions are provided.

According to some exemplary embodiments, a NF cycle comprises a reward anticipation block 246. In some embodiments, in the reward anticipation block, the patient interface is presented to the patient, and the patient is encouraged to modulate, for example to increase or decrease the amount of stimulating cues in the visual interface, while performing the task. In some embodiments, modulating the amount of stimulating cues in the patient interface indicates an increase in activity of the at least one mesolimbic region.

According to some exemplary embodiments, if the determined activity of the at least one mesolimbic brain region is close to a target activity level, for example a desired activity level, but is still lower than the target activity level, a boosting signal is optionally delivered to the patient at block 248. In some embodiments, the boosting signal indicates that the activity level of the mesolimbic brain region is in a desired direction, and encourages the patient to make an additional effort in order to reach a target activity level and a success reward cue indicating reaching the target activity level.

According to some exemplary embodiments, if the determined activity of the at least one mesolimbic brain region reaches a target activity level, then a success reward cue is delivered to the patient, at WIN block 250. In some embodiments, the training phase which includes the reward anticipation block 246 until getting the success reward cue at WIN block 250 trains a patient to increase an activity of the mesolimbic brain region and to expect a reward cue at block 250, if reaching a target activity level. In some embodiments, during the reward anticipation phase, the patient receives a continuous feedback, for example a visual feedback with an increasing amount of stimulating cues indicating an increase in the activity of the mesolimbic brain region.

According to some exemplary embodiments, once receiving a success reward cue at block 250, the system determines whether an activity level of the mesolimbic brain region correlates with reward consumption, at block 252. In some embodiments, at block 252 the system determines whether the activity level of the mesolimbic brain region indicates that the patient recognizes or appreciates the positive experiences in receiving the success reward cue at block 250. In some embodiments, if the determined activity level correlates with reward consumption a positive indication, for example a visual positive indication is delivered to the patient while presenting the visual interface. In some embodiments, this reward consumption block 252 trains the patient to find and apply tasks, for example cognitive tasks, in a way that elevates a reward consumption response in the patient after receiving an award.

According to some exemplary embodiments, a following the WIN block 250, the patient is instructed to maintain a desired activity level of the mesolimbic brain region, as part of a “holding” block 254. In some embodiments, if the patient maintains the desired activity level for a predetermined time period, then a positive indication, for example a visual positive indication is delivered to the patient while presenting the visual interface. Optionally, the “holding” block 254 is repeated at least two times in each NF session. In some embodiments, the “holding” block 254 trains the patient to find and apply tasks, for example cognitive tasks, in a way that elevates a sustained reward response after receiving an award.

According to some exemplary embodiments, each of the blocks or any combination and/or order of the blocks can be repeated at least twice, or any number of times. Optionally, the holding block 254 is performed prior to the reward anticipation block 246.

### Exemplary System for Delivery of a Treatment

Reference is now made to FIG. 3, depicting a system for delivery of a NF treatment, according to some exemplary embodiments of the invention.

According to some exemplary embodiments, a system 302 comprises a control unit 304, a patient interface 306 and a supervisor interface 308. In some embodiments, the patient interface 306 and the supervisor interface are functionally coupled to a control circuitry 310 of the control unit 304. In some embodiments, the control unit 304 further comprises a memory 312 which stores at least one algorithm and/or a lookup table, to be used when determining an activity level of at least one mesolimbic brain region. Alternatively or additionally, the memory 618 comprises at least one EFP, for example an EFP model of the at least one mesolimbic brain region, for example the EFP model described in WO2021260697A1, incorporated herein as a reference in its entirety. In other embodiments, the EFP model is stored on the EEG or on the computer.

According to some exemplary embodiments, the control unit 304 comprises an EEG recording unit 314 functionally coupled to the control circuitry 310. In some embodiments, the EEG recording unit 314 delivers signals from one or more electrodes positioned on a head of a subject to the control circuitry 310 for processing of the received signals and analysis of the processed signals in order to determine an activity level of the at least one mesolimbic brain region, and/or a relation between the determined activity and at least one reference value or indication thereof, stored in the memory 312. In some embodiments, the control circuitry 310 determines the activity level or the relation between the activity level and the at least one reference value or indication thereof, using the at least one of an algorithm, a lookup table and an EFP stored in the memory 312, for example as described in WO2021260697A1 or in WO2012104853A2, incorporated herein as a reference in their entirety.

According to some exemplary embodiments, the EEG recording unit is functionally coupled to at least one electrode, for example a plurality of electrodes 316 and 318 positioned on a head 612 of a patient 322. In some embodiments, the plurality of electrodes comprise 2, 3, 4, 5, 6, 7, 8, 9, 10 or any larger number of electrodes positioned and optionally attached to the head 320 of the patient 322. Optionally, the plurality of electrodes are arranged in an array. In some embodiments, the electrodes are positioned at specific locations on a head of the subject, for example at locations C3, C4, Cz, FCZ, P3, Pz and P4 of the extended 10-20 coordinate system. Alternatively, electrodes are positioned in any location or combination of locations of the extended 10-20 coordinate system. In some embodiments, the control unit 304 is connectable to at least one speaker or carphone 324 configured to deliver an audio signal to the patient 322.

According to some exemplary embodiments, the control unit comprises a communication circuitry 326 configured to receive and/or deliver signals, for example wireless signals to a remote device located outside the supervisor clinic, for example to a remote computer, a remote server or a remote cloud. In some embodiments, the remote device stores the at least one algorithm, lookup table and/or the EFP. In some embodiments, the control unit 304 transmits the electrical signals or processed electrical signals to the remote device via the communication circuitry 326 and receives via the communication circuitry 326 signals which indicate an activity level of the mesolimbic brain region and/or a relation between the activity level of the mesolimbic brain region and a reference value indicating a target activation level of the mesolimbic brain region.

According to some exemplary embodiments, the control circuitry 310 signals the patient interface 306 to display on a screen a visual interface and/or an audio interface and/or tactile and/or olfactory which continuously changes in response to an activation level or changes in the activation level relative to a baseline, of the mesolimbic brain region. In some embodiments, the interface is updated every 5 seconds, every 10 seconds, every 15 seconds, every 20 seconds, every 25 seconds or any intermediate, smaller or larger value, based on the signals received form the electrodes, for example electrodes 316 and 318. In some embodiments, the patient interface 306 changes the visual interface presented to the patient in a delay of about 5 seconds, about 10 seconds, about 15 seconds, about 20 seconds, about 25 seconds, or any intermediate, smaller or larger value, relative to the timing in which the electrical signal is received from the electrodes 316 and 318. In some embodiments, the patient interface comprises a display and/or speakers.

According to some exemplary embodiments, the patient interface is delivered in two dimension (2D), or in three dimension (3D). In some embodiments, the patient interface is delivered using virtual reality, augmented reality, with or without at least one of, audio, olfactory and tactile feedback.

According to some exemplary embodiments, the control circuitry 310 signals the supervisor interface to deliver visual and/or audio indications to a supervisor of the treatment. In some embodiments, the visual and/or audio indications present at least one of, a progress of the patient during the treatment, for example during the NF cycles 240, activity level of the mesolimbic brain region, changes in the activity level of the mesolimbic brain region, difference between a reference value and an activity level of the mesolimbic brain region, stage of the NF session, actions of the system and indications or cues delivered to the patient. Optionally, the supervisor interface 308 is a remote device of the supervisor coupled to the control unit 304 via the communication circuitry 326. In some embodiments, the control unit 304 transmits at least one of, data collected from the patient, data transmitted or displayed to the supervisor, to the remote device, for example for generating a database. In some embodiments, the dataset includes information collected from a plurality of systems and/or from a plurality of patients.

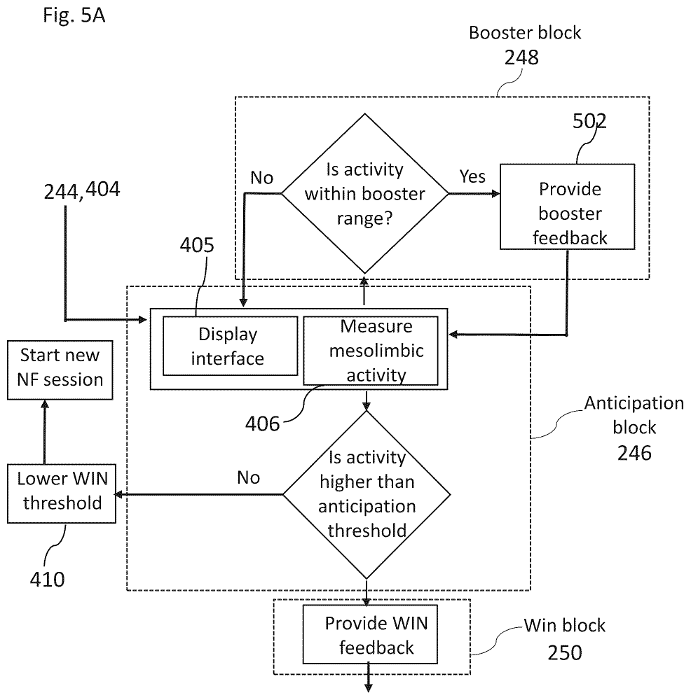

Exemplary System Activities Reference is now made to FIG. 4, depicting system 302 activities during a treatment session, for example during a NF session, according to some exemplary embodiments of the invention.

According to some exemplary embodiments, recordings are initiated at block 402. In some embodiments, recordings of signals by the electrodes, for example electrodes 316 and 318, is initiated at block 402. In some embodiments, the signals comprise EEG signals. In some embodiments, the signals received by the EEG recording unit 314, and are delivered to the control circuitry 310. In some embodiments, the recording of the signals is performed during the NF session.

According to some exemplary embodiments, instructions are delivered to the patient, at block 404 or before block 402 or later. In some embodiments, the instructions are delivered to the patient via the patient interface 306. In some embodiments, the instructions are delivered, for example as described at block 244 of FIG. 2C. in some embodiments the instructions can be in text format, audio format, video, a mix of all, or none at all.

According to some exemplary embodiments, a patient interface is displayed or in case of an audio/olfactory/tactile being delivered, to the patient, at block 405. In some embodiments, the patient interface comprises a dynamic scenario that is configured to change according to the activity level of at least one brain region, for example a mesolimbic brain region.

According to some exemplary embodiments, the order of blocks 402, 404 and 405 can be changed, for example instructions can be delivered at block 404 prior to initiation of recordings at block 402.

According to some exemplary embodiments, brain activity is determined at block 406. In some embodiments, determining a brain activity comprises determining an absolute brain activity level or a relative brain activity level relative to a reference value, for example a baseline. Alternatively or additionally, determining of brain activity comprises determining a change in brain activity relative to a baseline. In some embodiments, the brain activity is determined by the control circuitry or a remote device, as described in FIG. 3. In some embodiments, the baseline is used to calculate the brain activity, and a reference for example a threshold value is used to determine whether that brain activity crossed a certain level.

According to some exemplary embodiments, if the brain activity of the mesolimbic brain region increases, then the dynamic scenario presented to the patient is enriched with pleasant visual indications, as described for example in the reward anticipation block 246. In other embodiments the interface may become more depleted or undergo a change per the changes in the measured brain activity.

According to some exemplary embodiments, if the determined brain activity is higher, or in other embodiments lower, than a predetermined reference value indicating a threshold for a target activation level of the mesolimbic brain region, then a positive indication, for example a first positive indication is delivered to the patient at block 408. In some embodiments, the first positive indication is a high enrichment level of the visual interface. Alternatively, the first positive indication is a cue, for example a visual and/or an audio signal delivered to the patient during the delivery of the visual interface. In some embodiments, the first positive indication provides a success indication to the patient in reaching a target activation level of the mesolimbic brain region.

According to some exemplary embodiments, if after a while, the determined brain activity is lower than a predetermined threshold value, then the predetermined threshold value is optionally lowered at block 410. In some embodiments, the lower threshold value is used in the following NF session.

According to some exemplary embodiments, if the brain activity is higher than a predetermined threshold, then the predetermine threshold is elevated. In some embodiments, the elevated threshold is used in a following NF session.

Optionally, the system provides instructions to the patient to perform, for example apply a different task in order to modulate, for example increase or decrease the activation level of the mesolimbic brain region.

Optionally, if the determined brain activity is lower than the predetermined threshold value, but is within a predetermined activation level window of a boosting response, a stimulating visual indication is delivered to the patient, for example to encourage the patient to continue and perform the task he currently applies.

According to some exemplary embodiments, if the determined brain activity is higher than the predetermined threshold value, the threshold value is elevated at block 414. In some embodiments, the elevated threshold value is used in the following NF session.

According to some exemplary embodiments, the system identifies if a determined activation level of the mesolimbic brain region indicates reward consumption, at block 416. In some embodiments, the system identifies if the determined activation level indicates reward consumption, using the EFP and/or an algorithm, for example a P300 algorithm described for example in Giroldini W. et al., 2016, and in Stern Y. et al. (2016) stored in the memory 312. In some embodiments, if the system identifies that an activation level of the mesolimbic brain region indicates reward consumption, then a second positive indication is delivered to the patient at block 420. In some embodiments, the second positive indication is delivered to the subject in addition to the patient interface presented to the patient.

According to some exemplary embodiments, if the system identifies that an activation level of the mesolimbic brain region does not indicate reward consumption, then instructions are optionally provided to the patient to apply a different task or to change the way the task is applied, in order to achieve an activation level that indicates reward consumption.

According to some exemplary embodiments, the system optionally delivers an indication to the patient with instructions to maintain current activation level of the mesolimbic brain region (for example “holding”), at block 422. In some embodiments, the instructions are provided using the visual reference and include a time indication that counts the time that passed from providing the instructions or the time that remains until a predetermined time window ends.

According to some exemplary embodiments, the system determines if a determined brain activity level indicates a positive sustained reward response (holding), at block 423. In some embodiments, if the determined brain activity indicates a positive sustained reward response then a third positive indication is delivered to the patient at block 424.

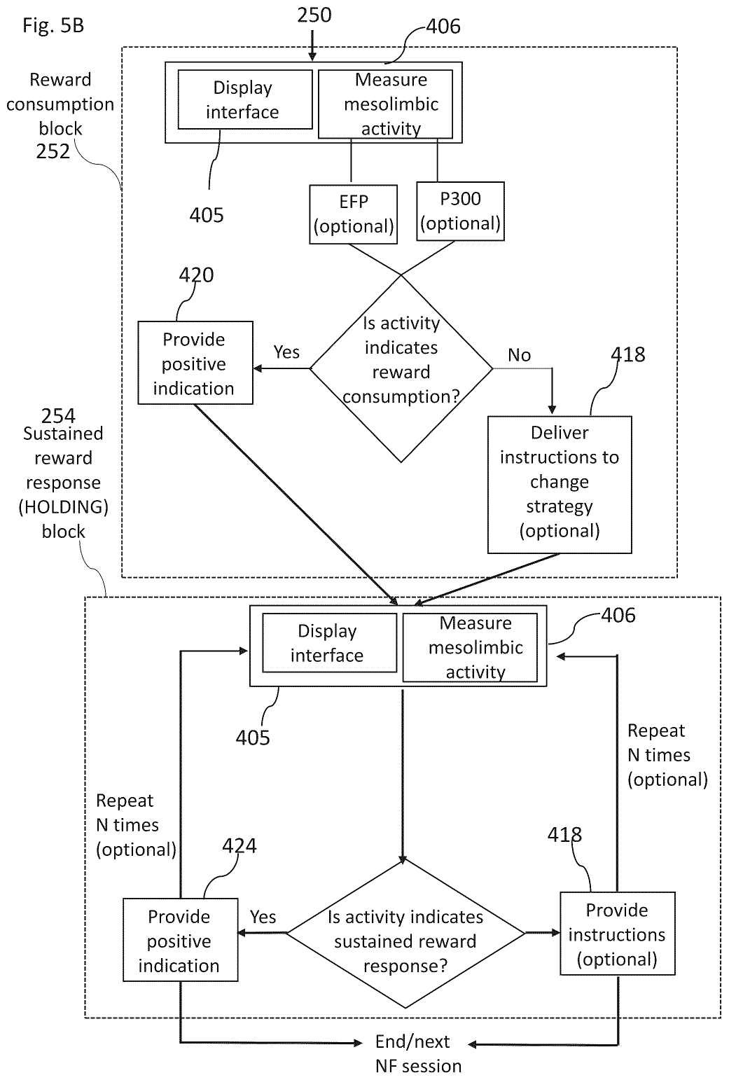

Reference is now made to FIGS. 5A and 5B depicting system activities divided to different blocks of the NF cycle, according to some exemplary embodiments of the invention.

According to some exemplary embodiments, instructions are provided to the patient, for example as described in blocks 244 described in FIG. 2C, and as described in block 404 described in FIG. 4.

According to some exemplary embodiments, during the anticipation block 246, a visual interface is displayed to the patient, for example as described at block 405, while activity of the at least one mesolimbic brain region is measured at block 406. In some embodiments, if the measured activity is within a booster range, then a booster feedback is delivered to the patient, at block 502. In some embodiments, a booster range of activity is an activity level window that is lower than a reference value indicating a target activation level of the mesolimbic brain region, for example a window located in a range between 40%-98% of the threshold value.

According to some exemplary embodiments, if the activity level of the mesolimbic brain region is higher than a predetermined threshold value, then a WIN feedback indication is delivered to the patient at the WIN block 250. Alternatively, if the activity level of the mesolimbic brain region is lower than the predetermined threshold value, the threshold value, for example a threshold for receiving a WIN feedback, is lowered at block 410.

According to some exemplary embodiments, during the reward consumption block 252, the system determines if the measured mesolimbic activity indicates reward consumption pattern, for example using EFP and/or P300 algorithm. In some embodiments, if activity indicates reward consumption a positive indication is delivered to the user. In some embodiments, if the activity does not indicate reward consumption, then instructions are optionally delivered to the patient at block 418.

According to some exemplary embodiments, and as explained in FIG. 2C and in FIG. 4, during a holding block 254, the patient is instructed to maintain a desired activity level of the mesolimbic brain region during a predetermined time period. In some embodiments, if the measured activity level of the mesolimbic brain region indicates a sustained reward response, then a positive indication is delivered to the patient at block 424. In some embodiments, if the measured activity level of the mesolimbic brain region does not indicate a sustained reward response (holding), then instructions are optionally provided to the patient to change or modify a strategy, for example a task, in order to reach a desired activity level indicating a sustained reward response. In some embodiments, the feedback provided at block 424 is continuously provided or is intermittently provided.

### Exemplary Patient Interfaces

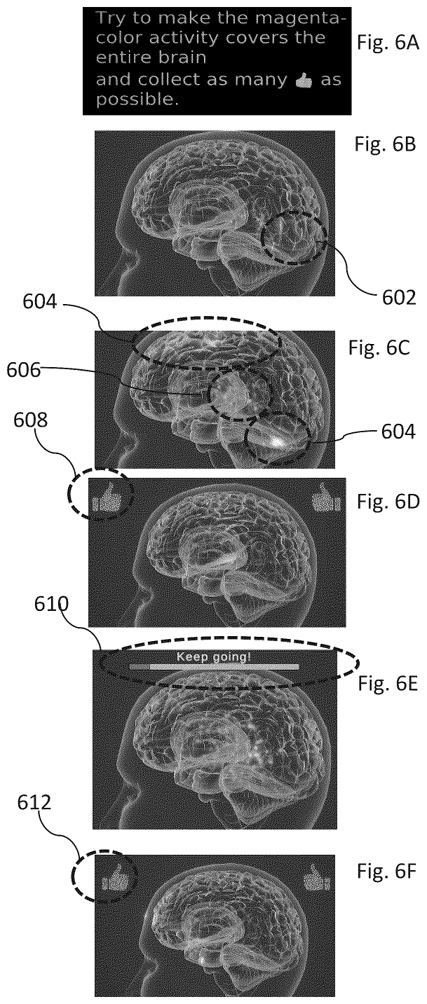

Reference is now made to FIG. 6A showing exemplary instructions provided to the patient, and to FIGS. 6B-6F showing changes in a brain interface during a NF session, according to some exemplary embodiments of the invention.

In some embodiments, FIG. 6A is an image of a visual interfaces which includes instructions to the patient.

In some embodiments, FIG. 6B is an image of a brain visual interface, for example during a reward anticipation block, where magenta color is limited and is concentrated in central regions 602 of the brain interface indicating a relatively low activation level of the brain, for example a relatively low activation level of at least one mesolimbic brain region, as determined at bock 406.

In some embodiments, FIG. 6C is an image of a brain visual interface where the magenta color has expanded and is now distributed throughout the brain at different brain regions 604, indicating an increase of the mesolimbic brain region activity. In addition, for example when a booster feedback is delivered to the patient, a series of colored spheres 606 is presented, and/or more regions of the brain are highlighted. In some embodiments, the booster feedback is configured to encourage the patient to proceed towards a reward signal. In some embodiments, when a reward signal, for example a WIN feedback is delivered, for example as described in FIG. 2C, the magenta color is widely distributed in the brain image.

In some embodiments, as shown in FIG. 6D, during reward consumption block, if an activity level of the mesolimbic brain region indicates reward consumption, then a visual indication is presented on the brain scheme. Same is true for the audio feedback.

In some embodiments, as shown in FIG. 6E, during a holding block 254, a time or a progress indicator 610 is presented to the patient, while magenta spots and colored spheres are distributed in the brain scheme to indicate a high activation level of the mesolimbic brain region.

In some embodiments, as shown in FIG. 6F, if the patient succeeded in maintaining high activation level, indicating a sustained reward response, then a visual indicator is presented on the brain scheme and a possible audio, tactile and/or olfactory feedback is delivered.

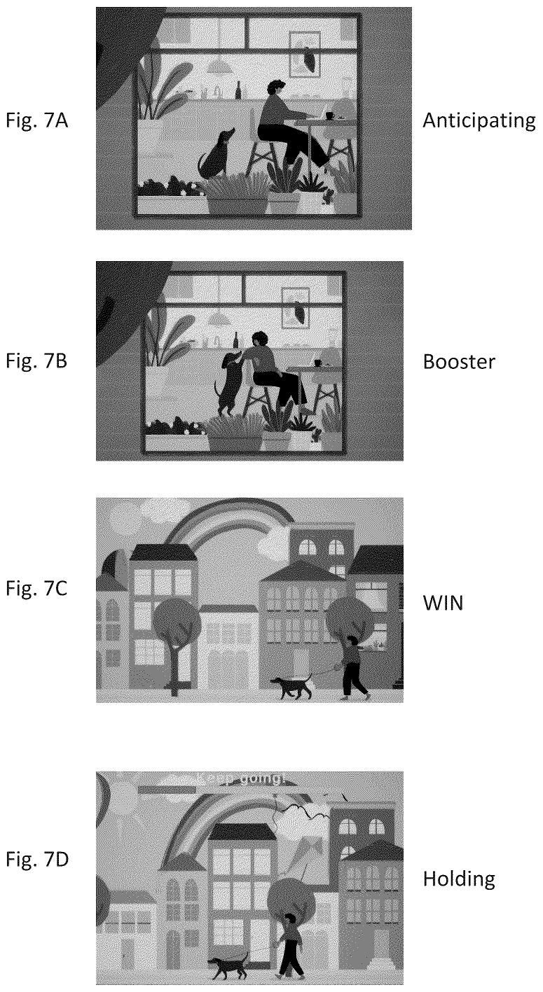

FIGS. 7A-7D show a different interface, and how the interface changes between the different blocks of the NF session described, for example, in FIG. 2C.

### Exemplary Supervisor Interface

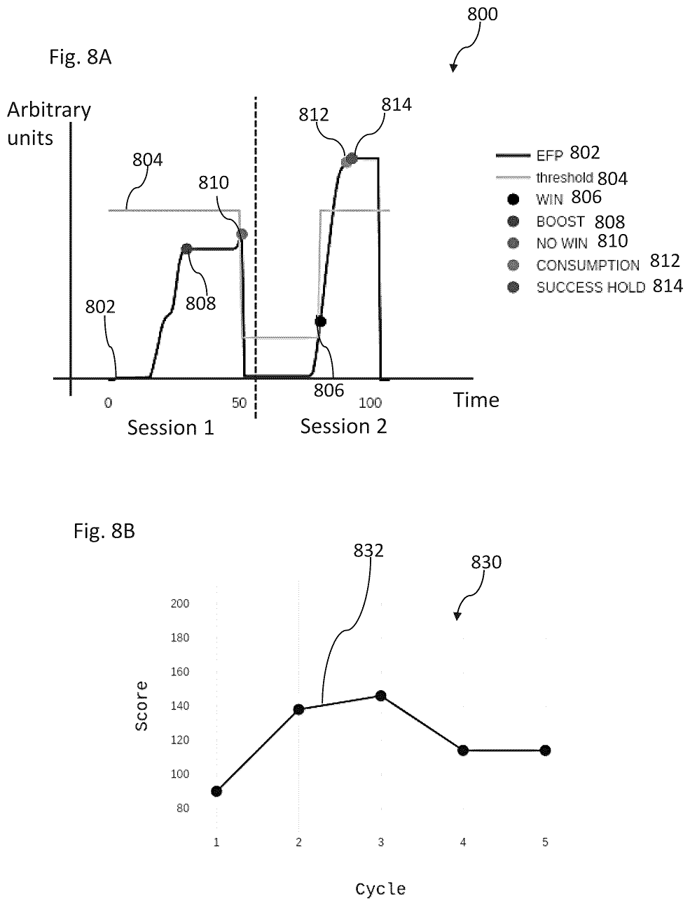

According to some exemplary embodiments, a supervisor interface is a visual interface presented to the supervisor of the treatment sessions, for example to monitor the progress of the patient during the NF sessions, and/or the different activities performed by the system, and/or review past performance. Reference is now made to FIG. 8A, depicting a visual interface of a NF session, according to some exemplary embodiments of the invention.

According to some exemplary embodiments, the visual interface 800 is displayed as a graph comprising an EFP line 802 indicting a change in a EFP of the mesolimbic brain region over time.

In some embodiments, the EFP line 802 represents accumulation of measurements over time, for example over a time period of up 30 30 seconds. In some embodiments, the change in EFP correlates with a determined activity or a change in a determined activity of the mesolimbic brain region, for example as described at block 406 in FIG. 4. In some embodiments, the graph further comprises a threshold line 804 indicating a predetermined value which is a threshold for receiving a WIN positive feedback following the anticipation block. In some embodiments, the threshold line 804 indicates a target activation level of the mesolimbic brain region.

According to some exemplary embodiments, as shown in the system delivered a booster indication to the patient at 808, when the EFP line is lower than the threshold line 804. In some embodiments, although he received a booster indication, for example the booster visual indication 606 in FIG. 6C, the EFP level remained below threshold in point 810 indicating no WIN signal. In some embodiments, and as shown in the graph, when reaching a no WIN point, the threshold 804 is lowered for the following NF session 2. In session 2, the EFP 802 level is higher than the threshold 804, and the system delivers a WIN feedback in 806. In some embodiments, when a WIN feedback is delivered, the threshold 804 is elevated for the following NF session. As shown in the graph, when the activity level EFP 802 indicates reward consumption, a positive indication of reward consumption 812 is delivered to the patient. In some embodiments, and as shown in the graph 800, when the activity level as indicated by the EFP line 802 remains high for a predetermined time period during the holding block, an indicator, for example indicator 612 shown in FIG. 6F, for success in holding 814 is delivered to the patient.

According to some exemplary embodiments, a visual interface presented to the supervisor includes information regarding the progress of the patient. In some embodiments, for example as shown in FIG. 8B, a graph 830 displays a change in a score received by the patient, indicated by line 832, between NF sessions. In some embodiments, the score is calculated based on the number of positive indicators the patient collected in each NF cycle, for example positive indicators for success in reward consumption and/or success in the holding block.

### Exemplary Experiment

An experiment was performed on a group of subjects diagnosed with MDD. In the experiment, the subjects, performed the NF treatment, described in this application according to a treatment plan that included performing of a total of 10 training sessions, during a time period of 5 weeks, in a frequency of 2 training sessions per week. The subjects underwent assessment prior to initiating the NF treatment, also termed herein as a NF training, prior to initiating the training, after 5 training sessions, and at the end of the NF training, after completing 10 sessions of the NF training. The assessment included the use of the Snaith-Hamilton pleasure scale modified for clinician administration (SHAPS-C), a Hamilton Depression Rating Scale (HDRS), and a Clinical Global Impression-Global Improvement (CGI-I) Scale, and self-filled questionnaires such as SHAPS-SR.

In the experiment and in some embodiments of the invention, the subjects diagnosed with MDD receive one or more medicines for depression, for example Selective Serotonin Reuptake Inhibitors (SSRI), Serotonin and Norepinephrine Reuptake Inhibitors (SNRI), Tricyclic and Tetracyclic Antidepressants, Atypical Antidepressants, Monoamine Oxidase Inhibitors (MAOIs), and N-methyl D-aspartate (NMDA) Antagonist. In the experiment and in some embodiments of the invention the subjects administer the one or more medicines before and/or after the NF training (NF treatment).

The Selective Serotonin Reuptake Inhibitors (SSRI) include one or more of, citalopram, escitalopram, paroxetine, fluoxetine, vortioxetine, vilazodone, and sertraline.

The Serotonin and Norepinephrine Reuptake Inhibitors (SNRI) include one or more of, duloxetine, venlafaxine, levomilnacipran, and desvenlafaxine.

The Tricyclic and Tetracyclic Antidepressants include one or more of, amoxapine, amitriptyline, maprotiline, desipramine, nortriptyline, doxepin, trimipramine, imipramine, and protriptyline.

The Atypical Antidepressants include one or more of, trazodone, bupropion, mirtazapine and nefazodone.

The Monoamine Oxidase Inhibitors (MAOIs) include one or more of, selegiline, isocarboxzaid, phenelzine, tranylcypromine.

The N-methyl D-aspartate (NMDA) Antagonist include esketamine.

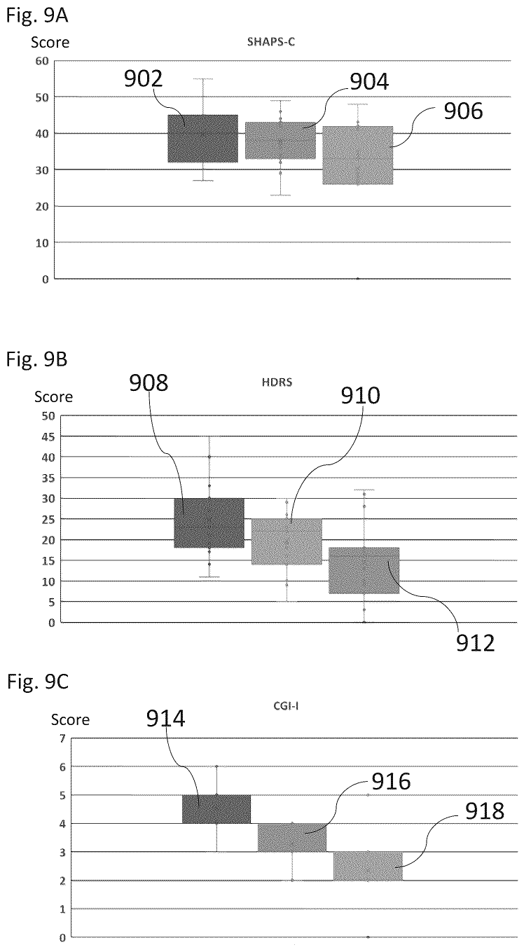

FIG. 9A is a graph showing a change in a score in the SHAPS-C scale as measured in 13 prior to the training (902), after 5 sessions (904), and after 10 sessions (906). As shown in FIG. 9A, the group of trainees achieved an average reduction of 1.93 points after 5 training sessions, and an average reduction of 3.46 points after 10 training sessions.

FIG. 9B is a graph showing a change in a score in the HDRS scale as measured in 13 subjects prior to training (908), after 5 sessions (910), and after 10 sessions (912). As shown in FIG. 9B, the group of trainees achieved an average reduction of 5.13 points after 5 training sessions, and an average reduction of 7.15 points after 10 training sessions.

FIG. 9C is a graph showing a change in a score in the CGI-I scale as measured in 13 subjects prior to the training (914), after 5 sessions (916) and after 10 sessions (918). As shown in FIG. 9C, the group of trainees achieved an average reduction of 1.27 points after 5 training sessions, and an average reduction of 1.77 after 10 training sessions.

According to the experiment, and according to some exemplary embodiments of the invention, a group of subjects diagnosed with MDD which performed the NF training described in the application, achieved a reduction of at least 3 points in average in the SHAPS-C scale, 5 weeks of the delivering of the NF training. Additionally or alternatively, the group of subjects achieved a reduction of at least 7 points in the HDRS scale, 5 weeks of the delivering of the NF training. Additionally or alternatively, the group of subjects achieved a reduction of at least 1.7 points in the CGI-I scale, 5 weeks of the delivering of the NF training.

As used herein with reference to quantity or value, the term “about” means “within +10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers there between.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for training a subject expected to experience or is experiencing at least one symptom of depression, comprising:
selecting a subject expected to experience in the future or is experiencing at least one symptom of depression;
training said selected subject using a neurofeedback (NF) training protocol to increase an activity of the brain positive valence system, said training comprises:
presenting to said selected subject a sensory indication instructing said subject to modify said presented sensory indication by performing at least one specific activity;
detecting during said presenting an increase in a brain pleasure response in said subject; and providing during said presenting to said subject a feedback signal indicating said increase in said brain pleasure response.

2. A method according to claim 1, wherein said at least one symptom of depression comprises depressed mood and/or anhedonia.

3. A method according to claim 1, wherein said at least one symptom of depression comprises at least one of, weight loss, weight gain, insomnia, hypersomnia, psychomotor agitation, psychomotor retardation, reduction in energy, worthlessness, guilt, concentration, suicidal thoughts and/or suicidal behaviors.

4. A method according to claim 1, wherein said selecting comprises selecting a subject having a first degree relative previously diagnosed with major depression or selecting a population of subjects which are a first responders.

5. A method according to claim 1, wherein said selecting comprises selecting a subject diagnosed with at least one of, post-traumatic stress disorder (PTSD), attention deficit hyperactivity disorder (ADHD), substance-induced mood disorder, chronic kidney disease (CKD) and/or Premenstrual dysphoric disorder (PMDD).

6. A method according to claim 1, wherein said selecting comprises selecting a subject in a risk for developing peripartum depression or already diagnosed with peripartum and/or postpartum depression.

7. A method according to claim 1, wherein said selecting comprises selecting a subject in a risk for developing seasonal affective disorder (SAD) or that was previously diagnosed with SAD, or a subject diagnosed with cancer.

8. A method according to claim 1, wherein said sensory indication comprises a visual and/or an audio indication, and wherein said providing comprises providing said feedback signal by modifying said visual and/or audio indication during said presenting.

9. A method according to claim 8, wherein said providing comprises providing said feedback signal by modifying said visual and/or audio indication in a way selected to increase said brain pleasure response in said subject.

10. A method according to claim 1, wherein said presented sensory indication is selected to increase a pleasure response in said subject and/or said at least one specific activity is configured to increase activity of said brain positive valence in said subject.

11. A method according to claim 1, wherein said detecting comprises measuring EEG signals from one or more electrodes positioned on a subject head during said presenting and said detecting, and wherein said detecting comprises detecting said increase in said brain pleasure response based on said measured EEG signals.

12. A method according to claim 1, wherein said detecting comprises detecting an increase in activity of at least one mesolimbic brain region.

13. A method according to claim 12, wherein said at least one mesolimbic brain region comprises a brain region related to a reward response in said subject.

14. A method according to claim 1, wherein said training comprises training said subject using said NF training protocol in two or more training sessions performed in a time difference of at least 12 hours therebetween.

15. A method according to claim 1, wherein said instructing comprises instructing said subject to modify during said presenting said presented sensory indication by performing said at least one specific activity, and wherein said activity comprises a cognitive activity or a mental activity.

16. A method according to claim 1, comprising modifying said presented sensory indication or selecting a sensory indication to be presented to said subject, according to said selected subject and prior to said presenting.

17. A method to treat a predetermined patient population for major depression, the method comprising:
selecting a population of patients diagnosed with major depression;
delivering a NF treatment to said population;
achieving a reduction of at least 3 points in average in a Snaith-Hamilton pleasure scale modified for clinician administration (SHAPS-C) in said selected population, after a time period of at least 5 weeks of said delivering.

18. A method according to claim 17, wherein said delivering comprises training said selected population using said NF treatment to increase a pleasure response in the brain and/or to increase activity of a positive valence system of the brain, when exposed to at least one sensory indication.

19. A method according to claim 17, wherein said delivering comprises training said selected population using said NF treatment to modulate an activity of at least one mesolimbic brain region or at least one biomarker thereof, when exposed to at least one sensory indication.

20. A method according to claim 19, wherein said delivering said NF treatment comprises:
recording EEG signals from said selected population during said exposure to said at least one sensory indication, extracting one or more signals which selectively indicate an activity level of said at least one mesolimbic brain region or biomarker thereof, from said recorded EEG signals, and providing feedback to said population regarding an activity of said at least one mesolimbic brain region, or said at least one biomarker thereof, based on the one or more extracted signals.

21. A method according to claim 17, wherein said delivered NF treatment is a functional magnetic resonance imaging (fMRI) neurofeedback treatment.

22. A system for delivery of a neurofeedback (NF) training, comprising:
a trainee interface configured to deliver at least one human detectable indication to a subject;
a control unit, comprising:
a memory, wherein said memory stores at least one NF training protocol for training said subject to increase activity of a brain positive valence system in said subject;
an EEG recording unit, configured to receive electrical signals from one or more electrodes positioned on a head of a subject;
a control circuitry, wherein said control circuitry is configured to:
signal said trainee interface to deliver a sensory indication stored in said memory to a subject, wherein said sensory indication is selected to increase a pleasure response in said subject brain;
measure EEG signals according to the electrical signals received by said EEG recording unit;
detect an increase in activity of at least one mesolimbic brain region based on said measured EEG signals using at least one algorithm stored in said memory, wherein said detected increase in activity indicates an increase in said pleasure response in said subject;
modify said sensory indication in a way selected to further increase said pleasure response in said subject; and
signal said trainee interface to deliver said modified sensory indication to said subject.

23. A system according to claim 22, wherein said control circuitry is configured to deliver a human detectable indication to said subject using said trainee interface with instructions to modify said sensory indication.

24. A system according to claim 22, wherein said sensory indication comprises a visual and/or audio interface.

25. A system according to claim 22, wherein said at least one mesolimbic brain region comprises at least one of, a ventral striatum (VS), a Nucleus Accumbens, a Ventral tegmental area, an Orbitofrontal cortex, and an Insula of the subject.

26. A system according to claim 22, wherein said detected increase in activity indicates an increase in activity of a brain positive valence system in said subject.

27. A system according to claim 22, wherein said control circuitry is configured to:
determine an activity level of said at least one mesolimbic brain region based on said measured EEG signals;
signal said trainee interface to deliver at least one feedback signal to said subject indicating that said determined activity level has reached a target activation level and that the target activation level is maintained for a pre-determined time period.
